(19) United States
(12) Patent Application Publication
(10) Pub. No.: US 2025/0255986 A1
(43) Pub. Date: Aug. 14, 2025

(54) DNA REVERTASE

(71) Applicants: Christopher Bradley, New York, NY (US); George McDonald Church, Brookline, MA (US); Mohamed Samin SHARIFI, Jena (DE)

(72) Inventors: Christopher Bradley, New York, NY (US); George McDonald Church, Brookline, MA (US); Mohamed Samin SHARIFI, Jena (DE)

(21) Appl. No.: 19/114,958

(22) PCT Filed: Nov. 1, 2022
(86) PCT No.: PCT/US2022/048617

§ 371 Date: Mar. 25, 2025

(51) Int. Cl. A61K 48/00; C12N 9/22; C12N 15/11; C12N 15/90

(52) U.S. Cl. CPC A61K 48/005; C12N 9/226; C12N 15/111; C12N 15/90; C12N 2310/20

(57) ABSTRACT

Disclosed are compounds and methods for changing a DNA sequence of an organism comprising delivering to the organism a change agent material which includes a strand invasion agent and a comparison agent. The strand invasion agent is configured to cause a separation of a double-stranded DNA strand in an organism cell. The comparison agent is configured to bind to a first single-stranded organism DNA strand, by including material that presents a number and configuration of hydrogen bonds complementary to the first single-stranded organism DNA strand, except, intentionally, at one or more base pair locations. Accordingly, when the comparison agent material binds to the first single-stranded organism DNA strand and at least one mismatched base pair is indicated on the first single-stranded organism DNA strand as a result of the binding, the organism initiates a mismatch repair process.

### CROSS REFERENCE TO RELATED APPLICATIONS

The present application claims priority from the following U.S. Provisional Applications, the entire disclosures of which, including but not limited to any and all cited references, are incorporated herein by reference: U.S. Provisional Application Nos. 63/274,128 (filed Nov. 1, 2021) and 63/340,605 (filed May 11, 2022). In addition, the entire disclosure of U.S. patent application Ser. No. 16/092,761 (filed Oct. 11, 2018), including but not limited to all of its file history documents and any and all references cited therein (collectively referred to herein as “the '761 application”), is incorporated herein by reference.

### FIELD OF THE INVENTION

The present invention relates generally to systems and methods for genetic modification, and more particularly to systems and methods for editing DNA.

### BACKGROUND OF THE INVENTION

Ever since discovering the structure and function of DNA and successfully sequencing the genomes of humans and other animals, scientists have been able to slowly tease apart the extremely complex cause and effect relationships at the heart of life itself. The deeper one looks, however, the deeper the complexities and interconnections seem to become.

Diseases that epitomized the importance of genomics, such as cancer, have revealed themselves to be caused by much more than a simple on/off switch, or single base-pair mutation. Instead, we are discovering that cancer is caused by a wide spectrum of different cascading mutations and ultimately different and constantly evolving diseases. So far, the same has been shown to be the case with aging. While no one has discovered a definitive cause of aging in humans or other animals, the current evidence points to aging being an emergent phenomenon resulting from failures of a large number of different pathways and processes.

One thing, however, is clear: increases in the amount and rate of mutagenesis, whether by external or internal factors, contribute greatly to a large number of genetic pathologies. This is thought to encompass everything from cancer and degenerative disease, to aging itself.

Therefore, the integrity of the information contained in DNA is clearly of critical importance to the survival and viability of an organism. For this reason, many mechanisms have evolved since the beginning of life to protect and repair DNA from errors and damage. However, it has also been discovered that although these innate repair and regeneration processes are very robust, they are not perfect. Given enough time and enough replicative events, errors in DNA replication will occur. For example, Reference 1 in the Bibliography illustrates and describes certain DNA repair pathways in the human body and the genes of each pathway that are involved.

Errors that relate to the sequence of the DNA itself can be considered information errors, where certain base pairs in the sequence shift to other base pairs. For example, an A-T pair can become a G-C pair. What makes these forms of errors, called single nucleotide polymorphisms, especially difficult to reverse is that most repair mechanisms rely on chemical/conformational changes in the DNA to detect errors, or they rely on one of the two existing DNA strands to act as a template for comparison. The reason base pair changes, whether single or multiple, remain undetected is that they succeed in changing both strands of the DNA without creating any overt signs of error. They effectively introduce noise to the sequence.

Studies have shown that as humans age or due to pre-existing defects in error correction genes, these random errors begin to accumulate in cells, and can lead to the emergence of cancers. It is also hypothesized that these random mutations are a major causative factor in the aging process itself.

Accordingly, there is a need to reverse the damage and mutagenesis present in a cell's genome, which may help cure and prevent diseases that emerge from their accumulation.

### SUMMARY OF THE INVENTION

The aforementioned and other challenges are addressed by the present invention, which among other advantages provides for the editing of the DNA of an organism, including but not limited to editing to reverse accumulated DNA mutations in the organism, and accordingly is useful for the prevention and treatment of genomic diseases, including but not limited to those somatically acquired.

Key challenges addressed by the present invention include (1) each cell having a random number and location of somatic mutations; (2) the mutations are locally silent, i.e., they do not show obvious signs of error at the molecular level (e.g., mismatches, dimers, nicks, etc.) and therefore, unless the mismatch leads to downstream changes that are detectable or known, there is no automatic way to know where or if the mutation occurred, and further therefore, it not possible to use one of the DNA strands as a repair template; (3) the mutation rate of editing these errors must be lower than the natural growth of the mutation, i.e., the solution cannot introduce more mutations than are being removed; (4) the mutations must be removed simultaneously, because each cell in the body accumulates an average of 5000 somatic mutations as humans approach 80-90 years of life, and therefore any solution would need to be able to make multiple changes at once; and (5) it must be known to what DNA sequence the mutated DNA sequence should be changed back.

The present invention addresses these key challenges and allows for the editing of DNA and accordingly the reversal of accumulated somatic mutations in a cell.

### Revertase

The present invention discloses a novel DNA editor that is referred to sometimes herein, not by way of limitation but rather for purposes of convenience, as a “Revertase”. While not limited to this function, the Revertase reveals informatic errors in an organism's genome and in doing so causes the organism's own repair mechanisms to correct the errors. The Revertase will be discussed in connection with preferred methods of the present invention.

### Change Agent Material

Preferred embodiments of the present invention include a change agent material that cause an organism DNA sequence to be changed to a desired DNA sequence by causing a changing of one or both of the nucleotide bases at each of one or more location on the organism DNA sequence to the nucleotide bases at a corresponding location on the desired DNA sequence.

To accomplish this, the change agent includes a comparison agent, which is also sometimes referred to herein in some embodiments as a mismatch template guide (MTG). The change agent optionally, but preferably, includes a mismatch repair facilitation agent, which is also sometimes referred to herein in some embodiments as a revertase protein complex (RPC).

### Comparison Agent

As to at least some embodiments, generally speaking without limitation, the comparison agent (e.g., MTG) serves at least three pertinent purposes: (1) to bind to a specific segment of organism DNA, which is complementary to the MTG; (2) to surface any mismatches in the bound segment; and (3) to anchor the mismatch repair facilitation agent (e.g., RPC) so that it can react to any mismatches.

A non-limiting example of a proposed mechanism for the MTG is as follows:

(1) First the germline sequence of the organism is determined through sequencing. The germline corresponds to the sequence the organism was conceived with, that is, the “original” DNA sequence before somatic mutations began accruing.

(2) The total germline sequence is divided into segments corresponding to the desired guide sequence. The goal is to subdivide the entire derived germline sequence into guides. The guides created can be overlapping but must contain all of the sequence that is desired to be reverted. For example, the guide sequence corresponds to the search space of that sequence. (Mutations that are contained in sequences outside the guide sequence cannot be found and reverted.)

(3) The length of the guide sequence is variable but preferably is designed to maximize the ability to detect true mutations, as opposed to simply detecting a non-complementary sequence.

(4) The main mechanism of the guide is to bind to one of the DNA strands that contains a complementary sequence. Once the guide binds, it separates the two DNA strands and forms a Guide-DNA hybrid.

(5) The guide may be RNA, DNA, a combination of the two or a sequence of different chemical components other than RNA or DNA.

(6) The guide may be single-stranded (ss) double-stranded (ds) or multi-stranded (ms) e.g. triplex or more.

(7) At this stage one of two scenarios can occur: (a) Scenario A: The DNA and Guide bind perfectly. There are no mismatches present. In this scenario, nothing happens; the complex releases and no additional reactions occur (because no mismatches were present); or (b) Scenario B: The DNA and Guide bind but contain mismatches. In this scenario, a mismatch loop is generated between the guide and the DNA. This mismatch loop triggers the activity of the attached RPC. The RPC binds to the mismatch loop and triggers the innate mismatch repair (MMR) reaction in the organism, which then reverts the mismatch to the sequence in the guide template.

### Strand Bias and Methylation

In order to direct the mismatch repair to the correct segment (i.e., the targeted DNA and not the guide), the guide must be appropriately altered to bias the MMR reaction away from altering it versus the targeted DNA. This could include methylation or any other modification necessary to bias the strand repair to the correct segment.

### Mismatch Repair Facilitation Agent

Further as to at least some embodiments, generally speaking without limitation, the mismatch repair facilitation agent (e.g., RPC) can comprise one or more of the MMR pathway proteins connected together to form a protein complex. In addition, other proteins can also be included, such as, for example, CRISPR Caspases (e.g., CRISPR Cas9) as well as various knockout and modified versions, including deactivated Cas9 (dCas9) and nickase Cas9 (nCas9), Base Editors, Prime Editors, etc. The MMR proteins that form the RPC an include (but are not limited to) one or more of the following: hMutS∝, hMutSβ, hMutL∝, hMutLβ, hMutLγ, Exol, Polδ (an enzyme complex found in eukaryotes that is involved in DNA replication and repair), PCNA, RPA, HMGB1, RFC, DNA Ligase I, Cas9 RuvC domain (alone or in combination with a guide RNA), Cas9 HNH domain, single-strand DNA (ssDNA) nuclease, deactivated Cas9 (dCas9) fusion with one or more mismatch repair proteins, CRISPR (or other editor) variants that allow for search, selection and/or repair of somatic mutations.

MMR in humans leverages many different proteins. Several key stages are currently known (see, e.g., Table 2 of Reference 2 in the Bibliography): (1) Recognition of the mismatch within the repetitive duplex DNA by MutS proteins. (2) Recruitment of the enzymes that function to repair the lesion in the mismatched DNA. (3) Excision of the mismatch base or incorrect sequence. (4) Resynthesis of the DNA along the parental template strand by DNA polymerase.

### Guided MutSα

In certain embodiments, having a mismatch recognition protein in the near vicinity improves the speed and efficiency of the mutation repair. Therefore, preferably, MutSα is guided to the area that is being checked for mismatching by the PNA template. This aspect of the invention will be described in greater detail below.

### Strand Invasion Agent

The change agent optionally, but preferably, includes a strand invasion agent. In certain embodiments, the strand invasion agent includes the comparison agent.

In embodiments utilizing a strand invasion agent, additional preferred mechanisms can be used to achieve reversal of mutations in a genome back to germline and/or editing of a section of cellular DNA to a desired sequence. For example without limitation, there are several different approaches that can be taken (individually or in combination with one another in any permutation(s)) to achieve the desired editing results. What one or more have in common is that they need to (and are able to) solve for one or more discrete mechanistic steps. Non-limiting example steps are outlined as follows: (1) Strand invasion/nuclear DNA strand separation. (2) Complementary base-pairing of the targeting strand with the targeted nuclear DNA sequence. (3) Base-pairing (or similar binding or matching) of the comparison agent (e.g., MTG or also referred to herein as a “spell-check template strand”) with the free nuclear DNA strand. (4) Induction of mismatch between the spell-check template strand and the nuclear DNA strand, only in the presence of a point mutation. (5) Recognition of the mismatch by mismatch repair enzymes. (6) Repair of the mismatch by mismatch repair enzymes.

### Peptide Nucleic Acid (PNA)

The stand invasion agent can in certain embodiments use a PNA mismatch recognition mechanism that includes a template that can invade the DNA efficiently and binds to the organism DNA strand in a sequence-specific manner (in certain embodiments, sometimes referred to herein as a “mismatch template”). Upon binding, if there are no mutations, the template binds without any mismatch bubbles, there will be no repair or editing activity, and the template will dissociate again. On the other hand, when the sequence does not match on a certain location (e.g., due to a point mutation), there is a mismatch bubble formed between the mismatch template and the DNA strand. This mismatch bubble will be recognized by the endogenous MMR mechanism and will be repaired. Therefore, one of the mechanisms by which our system detects integrated mutations is through the formation of mismatch bubbles; and thereby includes pointing out the location to the cell's own MMR system.

In order to achieve this, a template that can efficiently invade DNA is used. For this purpose (and/or other reasons) it is preferable to use peptide nucleic acids (PNAs). As examples, one or more different forms of PNAs can be used (individually or in combination), which will be detailed below.

The strand invasion agent can be modified to have certain beneficial properties, including but not limited to properties that increase efficiency of the separation of the organism DNA. In certain embodiments, one or both of the strand invasion agent and the change agent material comprise a compound having a chemical structure that provides one or more such properties. Example structures will be detailed below.

Accordingly, the present invention allows for the editing of DNA and accordingly the reversal of accumulated somatic mutations in a cell. It is anticipated that by removing accumulated mutagenesis, the cell will cease to display any of the phenotypic traits associated with the mutagenesis, such as, for example, cancer, neurodevelopmental disorders, development disorders, and other disorders. Additionally, to the extent that accumulated mutagenesis contributes to cellular aging, its removal is anticipated to have a substantial phenotypic effect on age-related properties of the cells of the organism and the organs and systems to which the cells belongs.

Aspects of the present invention are described herein and in the accompanying figures; however, it should be understood that the specific elements and methods in the descriptions are merely examples of elements and methods that can be used to embody and/or implement the described features and processes, and that such broader range of elements and methods are part of the present invention. Therefore, the descriptions herein and in the accompanying figures should be read broadly and understood broadly with respect to such specific elements and methods so as to encompass other elements and methods of such and/or similar and/or related types and/or functions, now known or hereinafter developed, to gain a proper understanding of the broad scope of the present invention.

### BRIEF DESCRIPTION OF THE DRAWINGS

FIGS. 1-9 illustrate respective preferred change agent material constructs of preferred embodiments of the present invention and associated methods of use thereof.

FIG. 10-11 illustrate use of respective preferred mismatch repair facilitation agents of preferred embodiments of the present invention.

FIG. 12 illustrates a preferred chemical structure of a strand invasion agent of preferred embodiments of the present invention.

FIGS. 13A and 13B illustrate results of testing conducted on a change agent material construct of preferred embodiments of the present invention.

FIG. 14 illustrates a method of preferred embodiments of the present invention.

### DETAILED DESCRIPTION OF PREFERRED EMBODIMENTS

Preferred embodiments of the present invention will now be described in detail.

All embodiments disclosed herein are presented not by way of limitation but rather to provide understanding and clarity. Any and all discussed aspects of the present invention are preferable and should be understood to be non-limiting examples. Any and all described components of any disclosed structures are optional, and any and all steps of any described methods are optional and need not be performed at all or in any particular order.

Without limiting their meanings in the art, terms used herein are understood to have any meanings ascribed to them here as well as any meanings ascribed to them in the '761 application.

### Preferred Methods

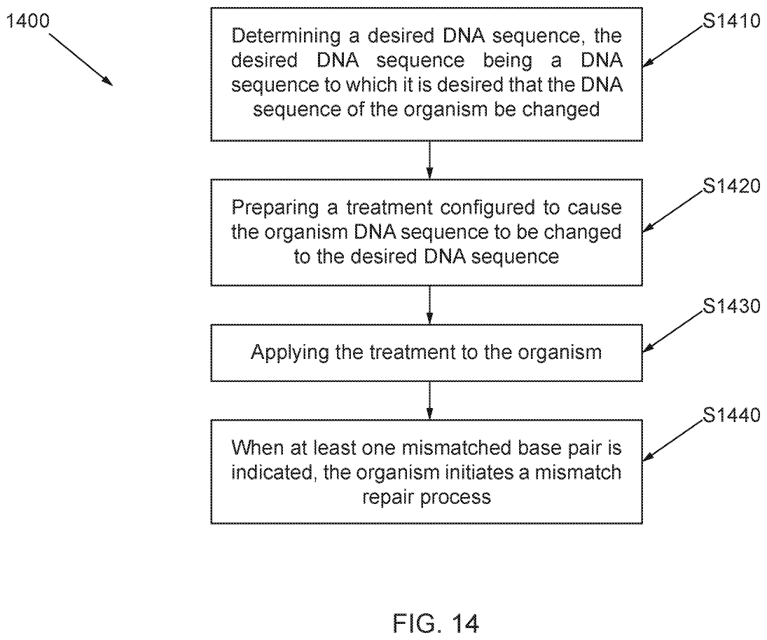

Referring now to FIG. 14, in preferred embodiments, the present invention comprises a method of changing a DNA sequence of an organism (e.g., method 1400 of FIG. 14).

Preferably, the organism DNA sequence comprises a first current sequence of nucleotide bases and a second current sequence of nucleotide bases matched by base pairing rules to the first current sequence of nucleotide bases to form a respective base pair of complementary nucleotide bases at each of one or more locations in the organism DNA sequence.

In preferred embodiments, the method comprises the steps of (1) determining a desired DNA sequence (e.g., S1410 of FIG. 14), (2) preparing a treatment configured to cause the organism DNA sequence to be changed to the desired DNA sequence (e.g., S1420 of FIG. 14), and (3) applying the treatment to the organism (e.g., S1420 of FIG. 14).

Preferably, the desired DNA sequence is a DNA sequence to which it is desired that the DNA sequence of the organism be changed.

Preferably, the desired DNA sequence comprises a first desired sequence of nucleotide bases and a second desired sequence of nucleotide bases matched by the base pairing rules to the first desired sequence of nucleotide bases to form a respective base pair of complementary nucleotide bases at each of one or more locations in the desired DNA sequence.

In preferred embodiments, the treatment is configured to cause the organism DNA sequence to be changed to the desired DNA sequence by causing a changing of one or both of the nucleotide bases at each of one or more of the organism DNA sequence locations to the nucleotide bases at a corresponding desired DNA sequence location.

In preferred embodiments, applying the treatment includes delivering to the organism at least one dose, and each dose includes respective change agent material that causes the changing.

In preferred embodiments, the change agent material includes a strand invasion agent and a comparison agent.

In preferred embodiments, the strand invasion agent is configured to cause a separation of a double-stranded DNA strand in at least one cell of the organism. Preferably, the double-stranded DNA strand includes the organism DNA sequence and comprises a first single-stranded organism DNA strand (e.g., including the first current sequence) and a second single-stranded organism DNA strand (e.g., including the second current sequence) bound to the first single-stranded organism DNA strand. Preferably, the separation causes an unbinding of the first and second single-stranded organism DNA strands.

In preferred embodiments, the comparison agent is configured to bind to the first single-stranded organism DNA strand. Preferably, the comparison agent is so configured at least in part by including material that presents a number and configuration of hydrogen bonds of at least a portion of the second desired sequence.

Accordingly, in preferred embodiments, when the comparison agent material binds to the first single-stranded organism DNA strand and at least one mismatched base pair is indicated on the first single-stranded organism DNA strand as a result of the binding, the organism initiates a mismatch repair process (e.g., S1440 of FIG. 14).

Aspects of the above described preferred embodiments will now be discussed in connection with specific examples.

### Organism DNA and Desired DNA

The organism can be any organism. Without limiting its meaning in the art, an organism can be a human, a non-human imal, a plant, and/or any entity having a DNA sequence.

Preferably, the DNA sequence of the organism is at least a portion of (e.g., at least a sub-sequence of) a current whole-genome DNA sequence of the organism. However, it should be understood that in some embodiments, the DNA sequence of the organism is a DNA sequence that is all or substantially all of the whole-genome DNA sequence of the organism. It should be further understood that the system and method of the present invention also can be used to substantially change one or more other types of nucleic acid sequences, of any possible length.

Preferably, the desired DNA sequence is the portion (e.g., sub-sequence) of the organism's germline whole-genome DNA sequence corresponding to the portion (e.g., sub-sequence) of the organism's current whole-genome DNA sequence referred to above. However, it should be understood that in some embodiments, the desired DNA sequence is a DNA sequence that is at least a portion of, or all or substantially all of, one of a germline whole-genome DNA sequence of the organism, a pre-mutagenic whole-genome DNA sequence of the organism, a global average whole-genome DNA sequence of the organism, a whole-genome DNA sequence of another organism, and a whole-genome DNA sequence that is an intentionally modified version of a germline whole-genome DNA sequence of the organism.

In certain embodiments, where a goal may be to conduct edits of individual locations on the organism DNA sequence (e.g., leaving all other locations unchanged), the desired DNA sequences can be a DNA sequence differing from the organism DNA sequence with respect to only one base pair. It should be understood that multiplex editing of individual locations (whether separated or adjacent) on the organism DNA sequence can also be accomplished by establishing the desired DNA sequence to be a DNA sequence differing from the organism DNA sequence with respect to only the base pairs at such locations. It should also be understood that multiplexing can mean, without limitation, multiple errors detected with a single comparison sequence or also multiple comparison sequences working on the same cellular DNA simultaneously.

Any and all methods, whether now known or hereafter developed, of determining the desired DNA sequence, are contemplated for use with the present invention, including but not limited to those described in the '761 application.

### General Example

As discussed above, in preferred embodiments, the change agent material includes one or more of a strand invasion agent and a comparison agent. A general, non-limiting example of the use of the change agent material will now be presented.

Upon administration to the organism, the change agent material, by way of the strand invasion agent, effects a separation of a double-stranded DNA in a cell of the organism (e.g., into first and second single-stranded organism DNA strands), and by way of the comparison agent, causes the comparison agent material to bind to one of the single-stranded organism DNA strands (e.g., the first single-stranded organism DNA strand).

With regard to the binding, because the first single-stranded organism DNA strand, which includes the first current organism DNA sequence (referred to above in the description of the organism DNA sequence that is being changed) is exposed to the comparison agent material, which presents the same number and configuration of hydrogen bonds as the second desired DNA sequence (referred to above in the description of the desired DNA sequence to which the organism DNA sequence is being changed), the first single-stranded organism DNA strand binds to the comparison agent material according to base pairing rules.

If the binding is complete (e.g., each of the nucleotide bases of the first single-stranded organism DNA strand pair respectively with its corresponding location on the comparison agent material), then there is no indication to the organism of a mismatched base pair, and accordingly, there is no need for a mismatch repair process to be initiated.

However, if the binding is not complete (e.g., if any one or more nucleotide bases of the first single-stranded organism DNA strand each fails to pair respectively with its corresponding location on the comparison agent material), then there is an indication to the organism of a mismatched base pair at the location of the nucleotide base on the first single-stranded organism DNA strand. In response to the indication, the organism initiates a mismatch repair process to replace the incorrect nucleotide base with a correct nucleotide base.

After the incorrect nucleotide base has been replaced, the comparison material becomes unbound from the first single-stranded organism DNA strand, and the first single-stranded organism DNA strand re-binds with the second single-stranded organism DNA strand from which it had been separated, to form a partially repaired organism DNA sequence. Then, as a result of the re-binding, in which the corrected nucleotide base now faces a non-matching nucleotide base, there is indication to the organism of a mismatched base pair at the location of the facing nucleotide bases on the partially repaired organism DNA strand. In response to the indication, the organism initiates another mismatch repair process, this time to replace the non-matching nucleotide base with a matching, correct nucleotide base. Once this second mismatch repair process is complete, the organism DNA sequence matches the desired DNA sequence, as intended.

### Illustrated Examples

A variety of additional non-limiting examples of the change agent material and its use will now be presented in connection with the figures.

In certain embodiments, the strand invasion agent includes the comparison agent. In other embodiments, the strand invasion agent and the comparison agent are separate compounds or separate molecules. In some of such embodiments, the compounds or molecules are initially bound to one another and separate during use. In other of such embodiments the compounds or molecules are initially bound to one another and remain bound during use.

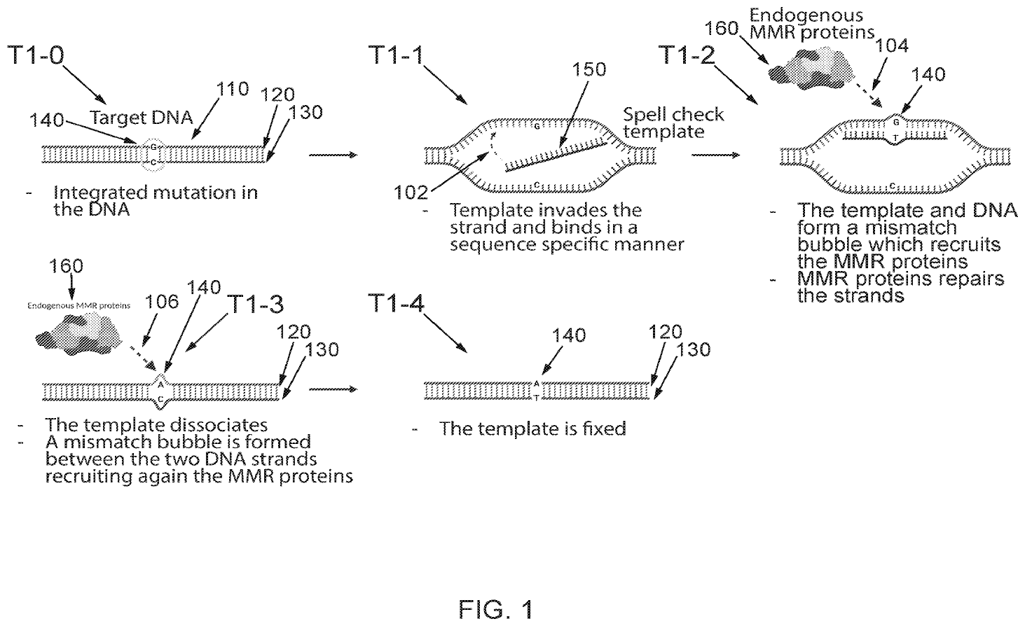

### FIG. 1

FIG. 1 illustrates a construct referred to herein not as limiting but for the sake of convenience, a basic PNA construct. In the example illustrated by FIG. 1, a PNA is the strand invasion agent and the comparison agent. Generally speaking, without limitation, in the example illustrated by FIG. 1, the change agent material comprises a PNA with a backbone having a sequence of nucleotides. This PNA can invade the DNA by binding to one of the DNA strands. If there is an integrated mutation in the DNA strand, there will be a mismatch “bubble” formed between the PNA strand and the DNA strand.

More particularly, in FIG. 1, at time T1-0, a double-stranded organism DNA strand 110 is represented by a first single-stranded organism DNA strand 120 and a second single-stranded organism DNA strand 130 bound to the first single-stranded organism DNA strand 120. At a location 140, nucleotide bases G and C are properly bound, but it has been determined that it would be desirable to change them to A and T, respectively. (For example, G and C may be present due to genetic mutation, and sequencing of the organism genome has indicated that in the germline DNA sequence of the organism, the nucleotide bases at this location are A and T, respectively.)

At time T1-1, change agent material of the present invention has been administered to the organism. In this example, the change agent material includes a PNA 150 having on a backbone a sequence of nucleotide bases complementary to a sequence of nucleotide bases on the first single-stranded organism DNA strand 120 that includes incorrect nucleotide base G at the location 140. The PNA 150 invades the double-stranded organism DNA strand 110 and binds to the first single-stranded organism DNA strand 120, as denoted by the dashed arrow 102.

At time T1-2, it can be seen that the PNA strand 150 is complementary to the DNA strand 120. However, at the location 140, the PNA strand 150 includes nucleotide base T. Due to the DNA strand 120 having nucleotide base G at the location 140, the binding of the PNA strand 150 to the DNA strand 120 causes a mismatch to be indicated at the location 140 on the DNA strand 120, as denoted by the bump surrounding nucleotide base G at the location 140. Further at time T1-2, the organism has initiated a mismatch repair process (as denoted by dashed arrow 104) in which endogenous MMR proteins 160 target the nucleotide base G and replace it with nucleotide base A to complement nucleotide base T on the PNA strand 150.

At time T1-3, it can be seen that the PNA strand has released or otherwise become unbound from the DNA strand 120 and the first and second single-stranded organism DNA strands 120,130 have paired to re-form the double-stranded organism DNA strand 110, except that at the location 140, there is a mismatch due to the nucleotide base A on the first single-stranded organism DNA strand 120 facing the nucleotide base C on the second single-stranded organism DNA strand 130. This causes a mismatch to be indicated at the location 140, as denoted by the bump surrounding the nucleotide bases A and C at the location 140. Further at time T1-3, the organism has initiated a mismatch repair process (as denoted by dashed arrow 104) in which endogenous MMR proteins 160 target the nucleotide base C on the second single-stranded organism DNA strand 130 and replace it with nucleotide base T to complement nucleotide base A on the first single-stranded organism DNA strand 120.

At time T1-4, it can be seen that the replacement is complete and the incorrect nucleotide bases G and C have been replaced with desired nucleotide bases A and T, respectively.

Accordingly, in this example illustrated by FIG. 1, the PNA 150 is the strand invasion agent and the comparison agent.

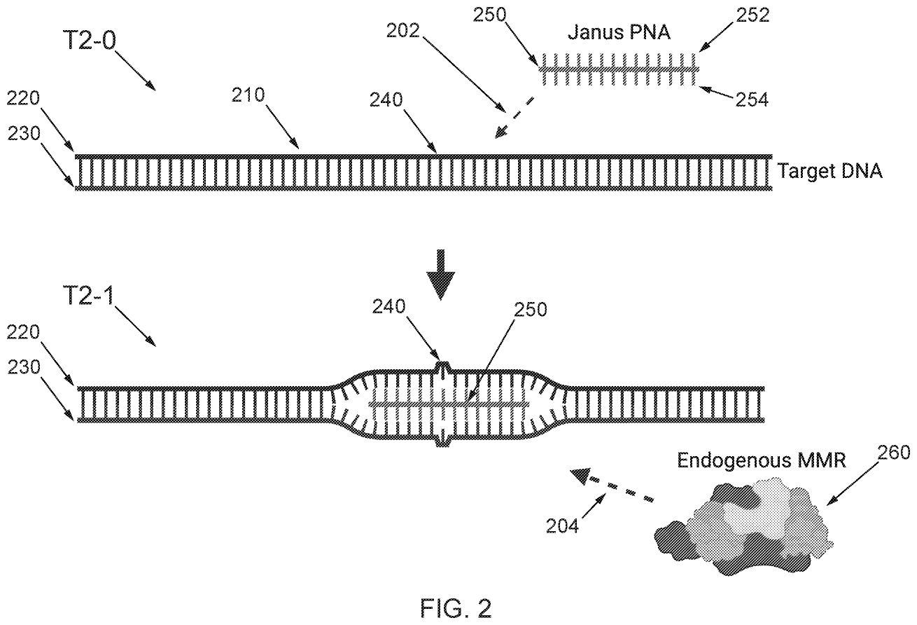

### FIG. 2

FIG. 2 illustrates a construct referred to herein not as limiting but for the sake of convenience, a Janus PNA construct. In the example illustrated by FIG. 2, a PNA is the strand invasion agent and the comparison agent. Generally speaking, without limitation, in the example illustrated by FIG. 2, the change agent material comprises a PNA with one backbone having nucleotides on two sides. This PNA can invade the DNA by binding both single-stranded DNA strands to the respective base pairs on either side of the PNA. If there is an integrated mutation in the DNA, there will be mismatch “bubbles” formed between the PNA strands and both DNA strands (e.g., two mismatch “bubbles” are formed simultaneously).

More particularly, in FIG. 2, at time T2-0, a double-stranded organism DNA strand 210 is represented by a first single-stranded organism DNA strand 220 and a second single-stranded organism DNA strand 230 bound to the first single-stranded organism DNA strand 220. At a location 240, nucleotide bases are properly bound, but it has been determined that it would be desirable to change them.

Further at time T2-0, change agent material of the present invention has been administered to the organism. In this example, the change agent material includes a PNA 250 having on a backbone a first sequence of nucleotide bases 252 (complementary to a sequence of nucleotide bases on the first single-stranded organism DNA strand 220, except at the location 240), and on the backbone a second sequence of nucleotide bases 254 (complementary to the sequence of nucleotide bases on the second single-stranded organism DNA strand 230 matching the sequence of nucleotide bases on the first single-stranded organism DNA strand 220, except at the location 240). The PNA 250 invades the double-stranded organism DNA strand 210 (as denoted by the dashed arrow 202).

At time T2-1, it can be seen that the first sequence of nucleotide bases 252 has bound to the first DNA strand 220, and the second sequence of nucleotide bases 254 has bound to the second DNA strand 230. Further at time T2-1, it can be seen that the sequences 252,254 are each complementary to the corresponding DNA strands 220,230. However, because the facing nucleotide bases at the location 240 are not complementary, the binding causes a mismatch to be indicated at the location 240 on both DNA strands 220,230, as denoted by the bumps surrounding the location 240. Further at time T2-1, the organism has initiated a mismatch repair process (as denoted by dashed arrow 204) in which endogenous MMR proteins 260 target the location 240 to replace the nucleotide bases with complementary ones.

Accordingly, in this example illustrated by FIG. 2, the PNA 250 is the strand invasion agent and the comparison agent.

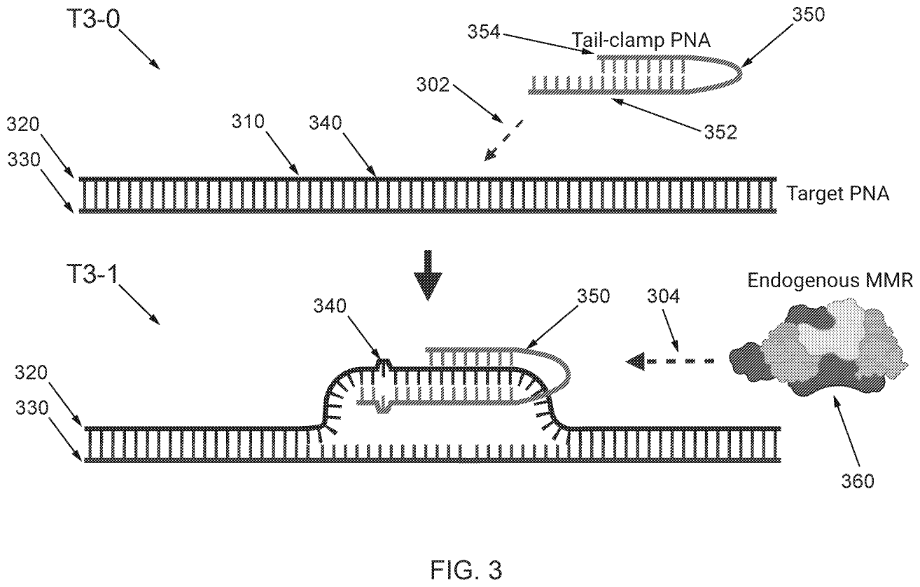

### FIG. 3

FIG. 3 illustrates a construct referred to herein not as limiting but for the sake of convenience, a tail-clamp construct. In the example illustrated by FIG. 3, a PNA is the strand invasion agent and the comparison agent. Generally speaking, without limitation, in the example illustrated by FIG. 3, the change agent material comprises a PNA that can bind DNA according to the base pairing rules and, in addition, by binding partially to the backbone in a sequence-specific manner forming, partially, a PNA:DNA:PNA triplex. The tail-clamp comparison construct is very stable, due to the triplex that is formed. The part that does not form a triplex but simply a PNA:DNA D-loop (P-loop) is the mismatch recognition part because mismatches in this part can be identified by the organism's MMR proteins.

More particularly, in FIG. 3, at time T3-0, a double-stranded organism DNA strand 310 is represented by a first single-stranded organism DNA strand 320 and a second single-stranded organism DNA strand 330 bound to the first single-stranded organism DNA strand 320. At a location 340, nucleotide bases are properly bound, but it has been determined that it would be desirable to change them.

Further at time T3-0, change agent material of the present invention has been administered to the organism (denoted by the arrow 302). In this example, the change agent material includes a PNA 350 having a tail-clamp formation, including a loop having a DNA binding section 352 and a backbone binding section 354. The DNA binding section 352 includes a sequence of nucleotide bases complementary to a sequence of nucleotide bases on the first single-stranded organism DNA strand 320, and the backbone binding section 354 includes a sequence that can bind to the backbone of the first single-stranded organism DNA strand 320.

The binding section 354 sequence is preferably a sequence of PNA that preferentially binds to the major groove in the DNA backbone as opposed to the DNA sequence. This “tail clamp” wraps around the DNA on the back and increases the stability of the PNA binding sequence overall.

At time T3-1, it can be seen that the sequence of nucleotide bases of the DNA binding section 352 has bound to the complementary to a sequence of nucleotide bases on the first DNA strand 320, and the binding section 354 sequence has bound to the backbone of the first DNA strand 320. Further at time T3-1, it can be seen that the facing sequences are complementary, except at location 340, which causes a mismatch to be indicated at the location 340 on the first DNA strands 320, as denoted by the bumps surrounding the location 340. Further at time T3-1, the organism has initiated a mismatch repair process (as denoted by dashed arrow 304) in which endogenous MMR proteins 360 target the location 340 to replace the nucleotide base on the first DNA strand with a complementary one.

Accordingly, in this example illustrated by FIG. 3, the PNA 350 is the strand invasion agent and the comparison agent.

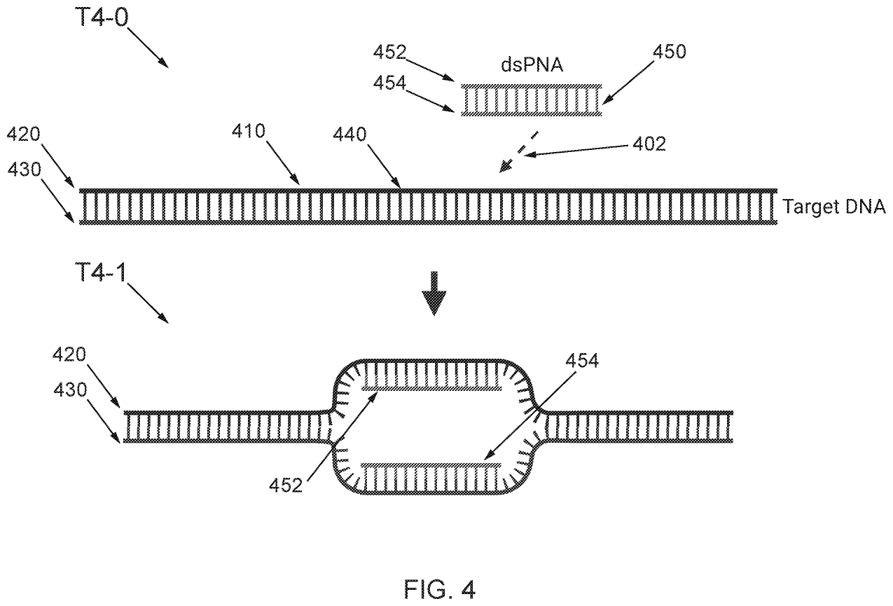

### FIG. 4

FIG. 4 illustrates a construct referred to herein not as limiting but for the sake of convenience, a dsPNA/PNA construct. In the example illustrated by FIG. 4, a PNA is the strand invasion agent and the comparison agent. Generally speaking, without limitation, in the example illustrated by FIG. 4, the change agent material comprises two PNA strands that are bound together according to the base-pairing rules. Pseudo-complementary base pairs (that prefer to bind to DNA rather than PNA) are used. Therefore, upon reaching the target site, both PNA strands invade the DNA and bind to their complementary DNA strand. Thus, both strands are simultaneously checked for mismatches.

More particularly, in FIG. 4, at time T4-0, a double-stranded organism DNA strand 410 is represented by a first single-stranded organism DNA strand 420 and a second single-stranded organism DNA strand 430 bound to the first single-stranded organism DNA strand 420. At a location 440, nucleotide bases are properly bound, but it has been determined that it would be desirable to change them.

Further at time T4-0, change agent material of the present invention has been administered to the organism (denoted by the arrow 402). In this example, the change agent material includes a double-stranded PNA 450 in which two single-stranded PNA strands 452,454 are bound together, each having a backbone and a respective sequence of nucleotide bases complementary to a respective one of the single-strand organism DNA strands 420,430.

At time T4-1, it can be seen that the PNA 450 has invaded the DNA 410 and each of the PNA strands 452,454 has bound to a respective one of the DNA strands 420,430. Further at time T4-1, it can be seen that the facing sequences are complementary. However, while not shown, at location 440 the bases of the DNA strands 420,430 are not complementary to the bases of the facing PNA strands 452,454, which causes a mismatch to be indicated at the location 440 on the DNA strands 420,430. Further at time T4-1, while not shown, the organism will in response initiate a mismatch repair process in which endogenous MMR proteins target the location 440 to replace the nucleotide base on the DNA strands with complementary ones.

Accordingly, in this example illustrated by FIG. 4, the PNA 450 is the strand invasion agent and the comparison agent.

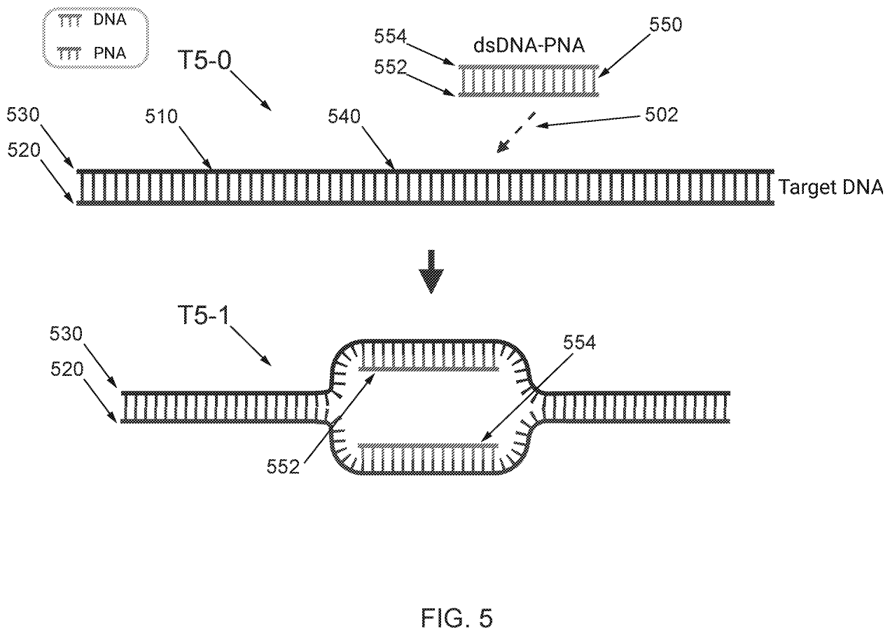

### FIG. 5

FIG. 5 illustrates a construct referred to herein not as limiting but for the sake of convenience, a dsPNA/RNA or dsPNA/DNA construct. In the example illustrated by FIG. 5, a PNA is the strand invasion agent and an RNA or a DNA is the comparison agent. Generally speaking, without limitation, in the example illustrated by FIG. 5, the change agent material comprises a PNA/RNA or PNA/DNA hybrid double-strand. The PNA does strand invasion while the DNA or RNA portion acts as a mismatch comparison agent (while discussed hereinbelow as applying to a PNA/RNA construct, this also applies to a PNA/DNA construct). First, the PNA invades the organism DNA, occupying one strand and forming a P-loop. The now single-stranded RNA part can bind to the unoccupied strand and screen for the mismatch.

More particularly, in FIG. 5, at time T5-0, a double-stranded organism DNA strand 510 is represented by a first single-stranded organism DNA strand 520 and a second single-stranded organism DNA strand 530 bound to the first single-stranded organism DNA strand 520. At a location 540, nucleotide bases are properly bound, but it has been determined that it would be desirable to change them.

Further at time T5-0, change agent material of the present invention has been administered to the organism (denoted by the arrow 502). In this example, the change agent material includes a double-stranded PNA/DNA 550 in which a single-stranded PNA strand 552 and a single-stranded DNA strand 554 are bound together, each having a backbone and a respective sequence of nucleotide bases complementary to a respective one of the single-strand organism DNA strands 520,530.

At time T5-1, it can be seen that the single-stranded PNA strand 552 has invaded the DNA 510 and bound to the second DNA strand 530 while the single-stranded DNA strand 554 has bound to the first DNA strand 520. As noted above, the single-stranded PNA strand 552 binds to the second DNA strand 530 without causing a mismatch indication, and instead functions to ensure that the single-stranded DNA strand 554 is able to more efficiently bind to the first DNA strand 520 and cause a mismatch indication if one is warranted.

Accordingly, further at time T5-1, it can be seen that the facing sequences of the single-stranded DNA strand 554 and the first DNA strand 520 are complementary, except that, while not shown, at location 540 the bases of the DNA strands 554,520 are not complementary, which causes a mismatch to be indicated at the location 540 on the DNA strand 520. Further at time T5-1, while not shown, the organism will in response initiate a mismatch repair process in which endogenous MMR proteins target the location 540 to replace the nucleotide bases on the DNA strands 520,530 with complementary ones.

Accordingly, in this example illustrated by FIG. 5, the PNA 552 is the strand invasion agent and the DNA 554 is the comparison agent.

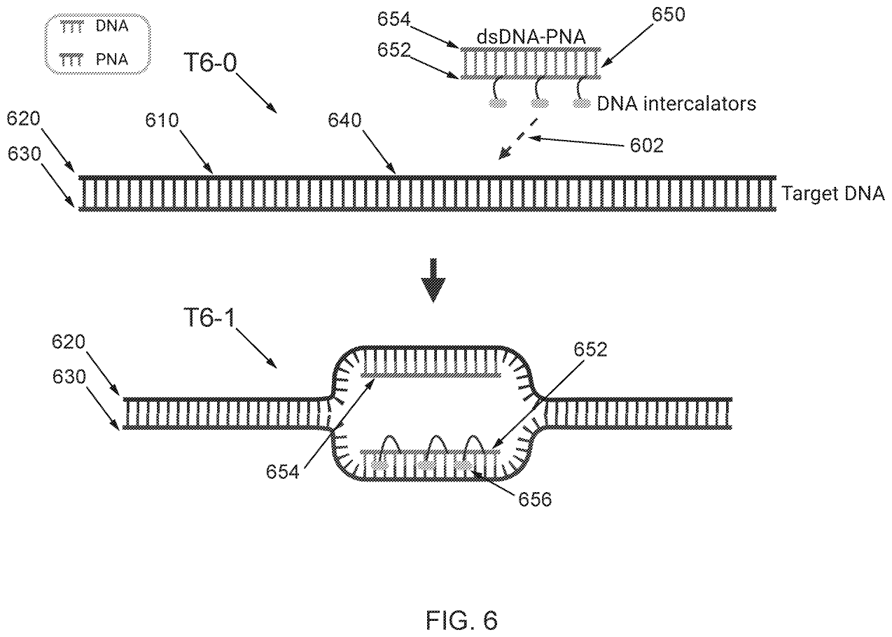

### FIG. 6

FIG. 6 illustrates a construct referred to herein not as limiting but for the sake of convenience, a dsPNA/RNA or dsPNA/DNA+intercalator construct. In the example illustrated by FIG. 6, a PNA is the strand invasion agent and an RNA or a DNA is the comparison agent. Generally speaking, without limitation, in the example illustrated by FIG. 6, the change agent material is the same in form and function as that in FIG. 5, except that the DNA/RNA portion carries an additional DNA intercalator, making the invasion and binding more efficient.

More particularly, in FIG. 6, at time T6-0, a double-stranded organism DNA strand 610 is represented by a first single-stranded organism DNA strand 620 and a second single-stranded organism DNA strand 630 bound to the first single-stranded organism DNA strand 620. At a location 640, nucleotide bases are properly bound, but it has been determined that it would be desirable to change them.

Further at time T6-0, change agent material of the present invention has been administered to the organism (denoted by the arrow 602). In this example, the change agent material includes a double-stranded PNA/DNA 650 in which a single-stranded PNA strand 652 and a single-stranded DNA strand 654 are bound together, each having a backbone and a respective sequence of nucleotide bases complementary to a respective one of the single-strand organism DNA strands 620,630. Further, the single-stranded DNA strand 654 further includes at least one intercalator 656.

At time T6-1, it can be seen that the single-stranded PNA strand 652 has invaded the DNA 610 and bound to the second DNA strand 630 while the single-stranded DNA strand 654 has bound to the first DNA strand 620. As noted above, the single-stranded PNA strand 652 binds to the second DNA strand 630 without causing a mismatch indication, and instead functions to ensure that the single-stranded DNA strand 654 is able to more efficiently bind to the first DNA strand 620 and cause a mismatch indication if one is warranted.

Accordingly, further at time T6-1, it can be seen that the facing sequences of the single-stranded DNA strand 654 and the first DNA strand 620 are complementary, except that, while not shown, at location 640 the bases of the DNA strands 654,620 are not complementary, which causes a mismatch to be indicated at the location 640 on the DNA strand 620. Further at time T6-1, while not shown, the organism will in response initiate a mismatch repair process in which endogenous MMR proteins target the location 640 to replace the nucleotide bases on the DNA strands 620,630 with complementary ones.

Accordingly, in this example illustrated by FIG. 6, the PNA 652 is the strand invasion agent and the DNA 654 is the comparison agent.

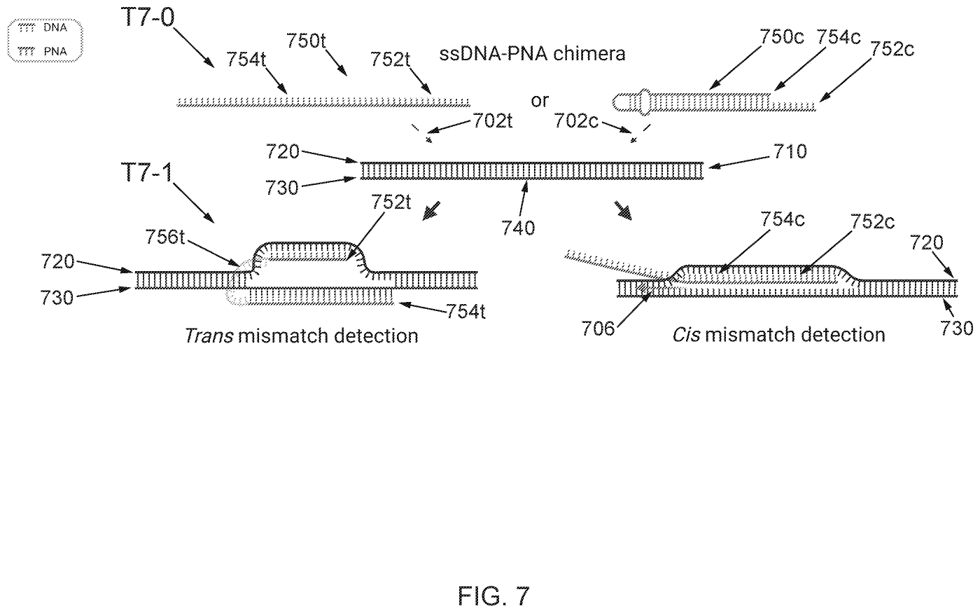

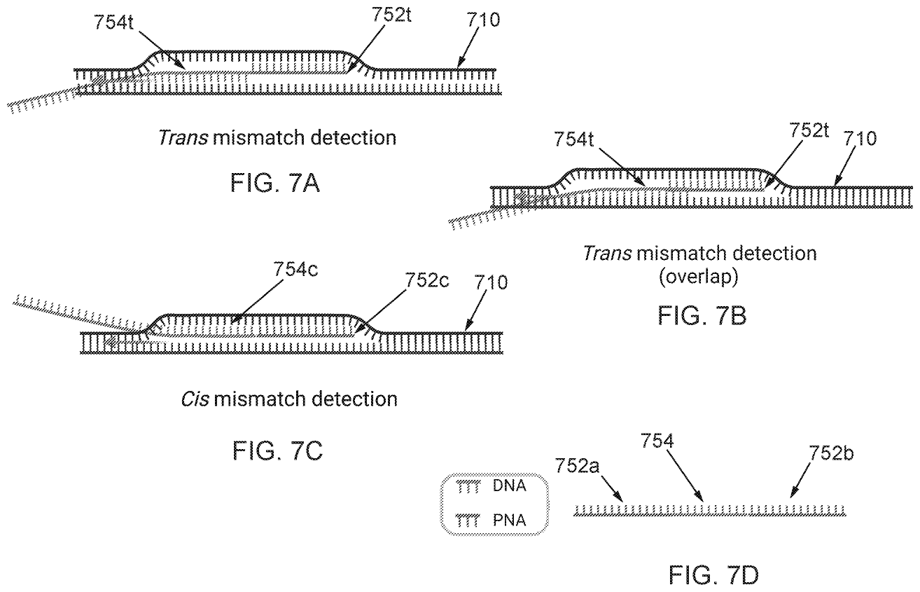

### FIG. 7

FIG. 7 illustrates a construct referred to herein not as limiting but for the sake of convenience, a PNA-RNA or PNA-DNA chimera. In the example illustrated by FIG. 7, a PNA is the strand invasion agent and an RNA or a DNA is the comparison agent. Generally speaking, without limitation, in the example illustrated by FIG. 7, the change agent material comprises a PNA strand attached to either an RNA or a DNA strand. The PNA and the DNA or RNA part have different functions: the PNA is used for the invasion of the target DNA, while the RNA or DNA part functions as the mismatch comparison agent. There are at least two ways to use the chimera template: by detecting mismatches in trans or in cis. Trans mismatch detection: In this detection, while the PNA invades one strand, the other part of the template (DNA or RNA) can check for mismatches on the unoccupied strand. Cis mismatch detection: In this detection, the PNA invades the DNA and zips it open; this chain reaction allows the DNA or RNA portion of the chimera template to bind to the free strand of the target DNA, inducing mismatch detection and mismatch repair.

More particularly, in FIG. 7, at time T7-0, a double-stranded organism DNA strand 710 is represented by a first single-stranded organism DNA strand 720 and a second single-stranded organism DNA strand 730 bound to the first single-stranded organism DNA strand 720. At a location 740, nucleotide bases are properly bound, but it has been determined that it would be desirable to change them.

Alternative sides of FIG. 7 will be discussed, one with reference to Trans mismatch detection and the other with reference to Cis mismatch detection.

With regard to Trans mismatch detection, further at time T7-0, change agent material of the present invention has been administered to the organism (denoted by the arrow 702t). In this example, the change agent material includes a single-stranded PNA-DNA strand 750t in which a single-stranded PNA strand 752t and a single-stranded DNA strand 754t are linked together in series, each having a backbone and a respective sequence of nucleotide bases complementary to a respective one of the single-strand organism DNA strands 720,730. (It can be seen illustrated at time T7-1 that the link 756t between the single-stranded PNA strand 752t and the single-stranded DNA strand 754t enables them to move away from one another while remaining linked. Preferably, the link is a covalent bond that is achieved through standard chemical reactions. It connects the peptide backbone of the PNA with the phosphate backbone of the DNA through a direct covalent bond.)

At time T7-1, it can be seen that the single-stranded PNA strand 752t has invaded the DNA 710 and bound to the second DNA strand 730 while the single-stranded DNA strand 754t has bound to the first DNA strand 720. The single-stranded PNA strand 752t binds to the second DNA strand 730 without causing a mismatch indication, and instead functions to ensure that the single-stranded DNA strand 754t is able to more efficiently bind to the first DNA strand 720 and cause a mismatch indication if one is warranted.

Accordingly, further at time T7-1, it can be seen that the facing sequences of the single-stranded DNA strand 754t and the first DNA strand 720 are complementary, except that, while not shown, at location 740 the bases of the DNA strands 754t,720 are not complementary, which causes a mismatch to be indicated at the location 740 on the DNA strand 720. Further at time T7-1, while not shown, the organism will in response initiate a mismatch repair process in which endogenous MMR proteins target the location 740 to replace the nucleotide bases on the DNA strands 720,730 with complementary ones.

Further with regard to Trans mismatch detection, FIGS. 7A and 7B illustrate that the PNA 752t and the DNA 754t in this example need not be linked, but rather the PNA 752t can be separate from the DNA 754t. With regard to FIG. 7A, once the PNA 752t separates the initial part of the organism DNA 710, it will be energetically easier to separate the neighboring areas. So if after invasion by the PNA 752t, a separate DNA 754t can invade the neighboring location in Trans. With regard to FIG. 7B, this does not need to happen immediately adjacent to the invading PNA 752t. It can instead be overlapping with the invading PNA 752t (for both the PNA:DNA chimera or PNA 752t and DNA 754t delivered separately).

With regard to Cis mismatch detection, further at time T7-0, change agent material of the present invention has been administered to the organism (denoted by the arrow 702c). In this example, the change agent material includes a single-stranded PNA-DNA strand 750c in which a single-stranded PNA strand 752c and a single-stranded DNA strand 754c are linked together in a loop, each having a backbone and a respective sequence of nucleotide bases complementary to a respective section of the single-stranded organism DNA strand 720.

At time T7-1, it can be seen that the single-stranded PNA strand 752c has invaded the DNA 710 and bound to a section of the first DNA strand 720 while the single-stranded DNA strand 754c has bound to a nearby section of the first DNA strand 720. The single-stranded PNA strand 752c binds to the first DNA strand 720 without causing a mismatch indication, and instead functions to ensure that the single-stranded DNA strand 754c is able to more efficiently bind to the first DNA strand 720 and cause a mismatch indication if one is warranted. The arrow 706 represents the DNA part of the DNA-PNA chimera continuing to separate the duplex DNA until the entire portion of the DNA is bound to either the same (Cis) or opposite (Trans) side. The PNA invades initially, but the arrow 706 shows that if the DNA and PNA are bound together, then once the PNA lands and starts to separate the strands then the DNA can continue that process (e.g., because it is less energy intensive than the initial separation). The arrow 706 shows the direction of that binding and separation.

Accordingly, further at time T7-1, it can be seen that the facing sequences of the single-stranded DNA strand 754c and the first DNA strand 720 are complementary, except that, while not shown, at location 740 the bases of the DNA strands 754c,720 are not complementary, which causes a mismatch to be indicated at the location 740 on the DNA strand 720. Further at time T7-1, while not shown, the organism will in response initiate a mismatch repair process in which endogenous MMR proteins target the location 740 to replace the nucleotide bases on the DNA strands 720,730 with complementary ones.

Further with regard to Cis mismatch detection, FIG. 7C illustrates that the PNA 752c and the DNA 754c in this example need not be linked, but rather the PNA 752c can be separate from the DNA 754c. With regard to FIG. 7C, once the PNA 752c separates the initial part of the organism DNA 710, it will be energetically easier to separate the neighboring areas. So if after invasion by the PNA 752c, a separate DNA 754c can invade the neighboring location in Cis.

Further with regard to both Trans and Cis mismatch detection, FIG. 7D illustrates that in certain constructs, the DNA 754 can be flanked by two PNAs (752a,752b), forming one long strand. The PNA flanks will be the invader, whereas the DNA will be the mismatch comparison agent. This configuration and similar configurations, for example without limitation, would in certain embodiments, allow a larger portion of the double-stranded DNA strand to be opened, since the invading PNA strands flanking the comparison agent DNA allow for more space for the comparison agent DNA to make a comparison. The separated organism DNA strands will not close between the flanked comparison agent DNA because they are very close to each other (only separated by the comparison agent DNA they are bound/ligated to).

Accordingly, in this example illustrated by FIG. 7, the PNA 752 is the strand invasion agent and the DNA 754 is the comparison agent.

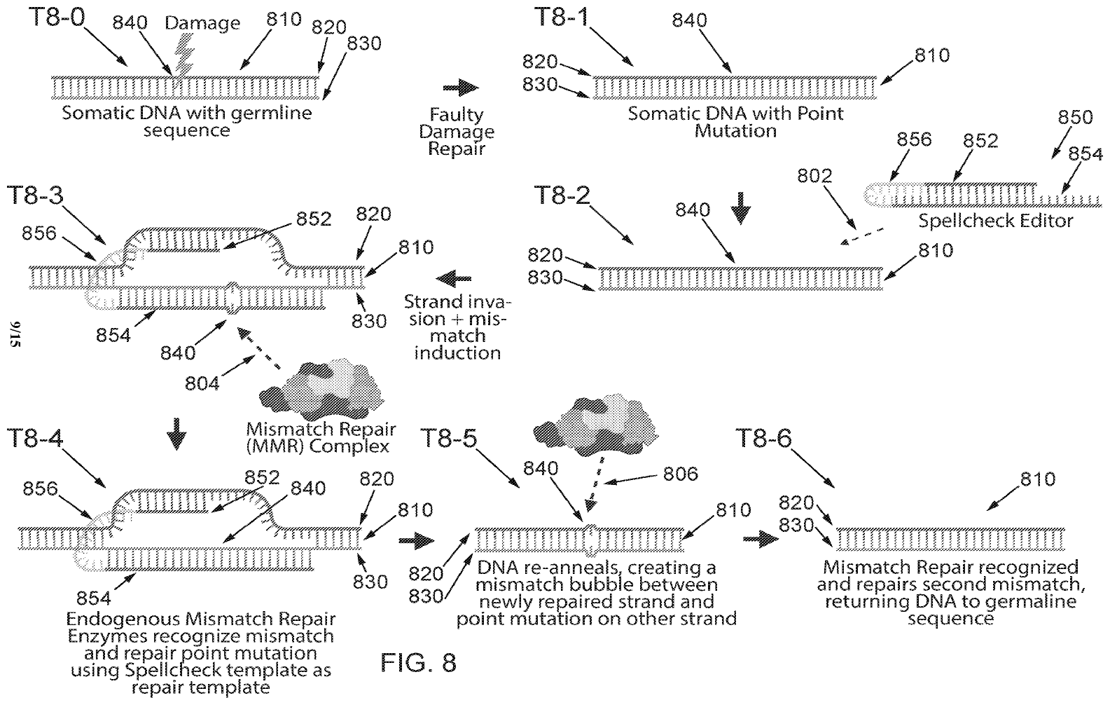

### FIG. 8

FIG. 8 illustrates a construct referred to herein not as limiting but for the sake of convenience, a PNA-DNA mechanism. In the example illustrated by FIG. 8, a PNA is the strand invasion agent and an RNA or a DNA is the comparison agent. Generally speaking, without limitation, in the example illustrated by FIG. 8, the change agent material comprises a PNA strand attached to either an RNA or a DNA strand. The PNA and the DNA or RNA part have different functions: the PNA is used for the invasion of the target DNA, while the RNA or DNA part functions as the mismatch comparison agent, binding to the free strand of the target DNA, inducing mismatch detection and mismatch repair.

More particularly, in FIG. 8, at time T8-0, a double-stranded organism DNA strand 810 is represented by a first single-stranded organism DNA strand 820 and a second single-stranded organism DNA strand 830 bound to the first single-stranded organism DNA strand 820. At a location 840, nucleotide bases are being damaged by radiation. At time T8-1, fault damage repair has led to a mutation at location 840, and it is desirable that the mutation be reverted.

At time T8-2, change agent material of the present invention has been administered to the organism (denoted by the dashed arrow 802). In this example, the change agent material includes a PNA-DNA strand 850 in which a single-stranded PNA strand 852 and a single-stranded DNA strand 854 are linked in series and bound to form a loop. Each has a backbone and a respective sequence of nucleotide bases complementary to a respective one of the single-strand organism DNA strands 820,830. It can be seen at time T8-2 and T8-3 that the link 856 between the single-stranded PNA strand 852 and the single-stranded DNA strand 854 enables them to move away from one another while remaining linked.

At time T8-3, it can be seen that the single-stranded PNA strand 852 has invaded the DNA 810 and bound to the second DNA strand 820 while the single-stranded DNA strand 854 has bound to the first DNA strand 830. The single-stranded PNA strand 852 binds to the second DNA strand 820 without causing a mismatch indication, and instead functions to ensure that the single-stranded DNA strand 854 is able to more efficiently bind to the first DNA strand 830 and cause a mismatch indication if one is warranted.

Accordingly, further at time T8-3, it can be seen that the facing sequences of the single-stranded DNA strand 854 and the first DNA strand 830 are complementary, except that at location 840 the bases of the DNA strands 854,830 are not complementary, which causes a mismatch to be indicated at the location 840 on the DNA strand 830. Further at time T8-3, the organism will in response initiate a mismatch repair process (denoted by dashed arrow 804) in which endogenous MMR proteins 860 target the location 840 to replace the nucleotide base on the DNA strand 830 with a complementary one.

At time T8-4, it can be seen that the nucleotide base on the DNA strand 830 has been replaced with a complementary one, while the corresponding nucleotide base on the DNA strand 820 remains incorrect.

At time T8-5, it can be seen that once the PNA-DNA strand 850 has dissociated, the first and second DNA strands 820,830 re-pair to re-form the double-stranded organism DNA strand 810, except that at the location 840, there is a mismatch due to the nucleotide base on the first single-stranded organism DNA strand 830 facing a now-incompatible nucleotide base on the second single-stranded organism DNA strand 820. This causes a mismatch to be indicated at the location 840, as denoted by the bump surrounding the nucleotide bases at the location 840. Further at time T8-5, the organism has initiated a mismatch repair process (as denoted by dashed arrow 806) in which endogenous MMR proteins 860 target the nucleotide base on the second single-stranded organism DNA strand 820 and replace it with a nucleotide base compatible with the nucleotide base on the first single-stranded organism DNA strand 830.

At time T8-5, it can be seen that the replacement is complete and the incorrect nucleotide bases have been replaced with desired nucleotide bases.

Accordingly, in this example illustrated by FIG. 8, the PNA 852 is the strand invasion agent and the DNA 854 is the comparison agent.

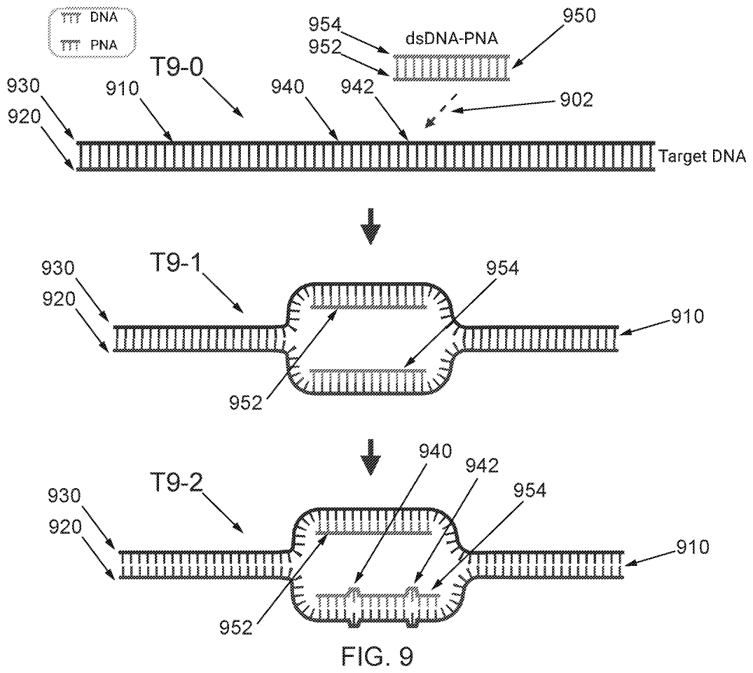

### FIG. 9

FIG. 9 illustrates a construct referred to herein not as limiting but for the sake of convenience, a multiple mismatch construct. In this example, the comparison DNA is inducing two simultaneous mismatches in the cellular DNA strand it is bound to. This shows the ability of the comparison sequence to detect multiple mismatches simultaneously in its area of comparison.

FIG. 9 illustrates a construct referred to herein not as limiting but for the sake of convenience, a multiple mismatch construct. In the example illustrated by FIG. 9, a PNA is the strand invasion agent and an RNA or a DNA is the comparison agent. Generally speaking, without limitation, in the example illustrated by FIG. 9, the change agent material comprises a PNA/RNA or PNA/DNA hybrid double-strand. The PNA does strand invasion while the DNA or RNA portion acts as a mismatch comparison agent (while discussed hereinbelow as applying to a PNA/RNA construct, this also applies to a PNA/DNA construct). First, the PNA invades the organism DNA, occupying one strand and forming a P-loop. The now single-stranded RNA part can bind to the unoccupied strand and screen for mismatches.

More particularly, in FIG. 9, at time T9-0, a double-stranded organism DNA strand 910 is represented by a first single-stranded organism DNA strand 920 and a second single-stranded organism DNA strand 930 bound to the first single-stranded organism DNA strand 920. At locations 940,942, nucleotide bases are properly bound, but it has been determined that it would be desirable to change them.

Further at time T9-0, change agent material of the present invention has been administered to the organism (denoted by the arrow 902). In this example, the change agent material includes a double-stranded PNA/DNA 950 in which a single-stranded PNA strand 952 and a single-stranded DNA strand 954 are bound together, each having a backbone and a respective sequence of nucleotide bases complementary to a respective one of the single-strand organism DNA strands 920,930.

At time T9-1, it can be seen that the single-stranded PNA strand 952 has invaded the DNA 910 and bound to the second DNA strand 930 while the single-stranded DNA strand 954 has bound to the first DNA strand 920. As noted above, the single-stranded PNA strand 952 binds to the second DNA strand 930 without causing a mismatch indication, and instead functions to ensure that the single-stranded DNA strand 954 is able to more efficiently bind to the first DNA strand 920 and cause mismatch indications if any are warranted.

At time T9-2, it can be seen that the facing sequences of the single-stranded DNA strand 954 and the first DNA strand 920 are complementary, except that at locations 940,942 the bases of the DNA strands 954,920 are not complementary, which causes two mismatches to be indicated (one at at each of the locations 940,942) on the DNA strand 920. Further at time T9-1, while not shown, the organism will in response initiate a mismatch repair process in which endogenous MMR proteins target the locations 940,942 to replace the nucleotide bases on the DNA strands 920,930 with complementary ones.

Accordingly, in this example illustrated by FIG. 9, the PNA 952 is the strand invasion agent and the DNA 954 is the comparison agent.

### Mismatch Repair Facilitation Agent

As discussed above, in certain embodiments, the change agent material includes a mismatch repair facilitation agent configured to facilitate the mismatch repair process.

Preferably, the mismatch repair facilitation agent includes one or more of the following molecules: hMutS∝, hMutSβ, hMutL∝, hMutLβ, hMutLγ, Exol, Pol δ, PCNA, RPA, HMGB1, RFC, DNA Ligase I, Cas9 RuvC domain (alone or in combination with a guide RNA), Cas9 HNH domain, single-strand DNA (ssDNA) nuclease, deactivated Cas9 (dCas9) fusion with one or more mismatch repair proteins, CRISPR (or other editor) variants that allow for search, selection and/or repair of somatic mutations.

These molecules are known to have beneficial effects on one or more processes involved in the MMR process.

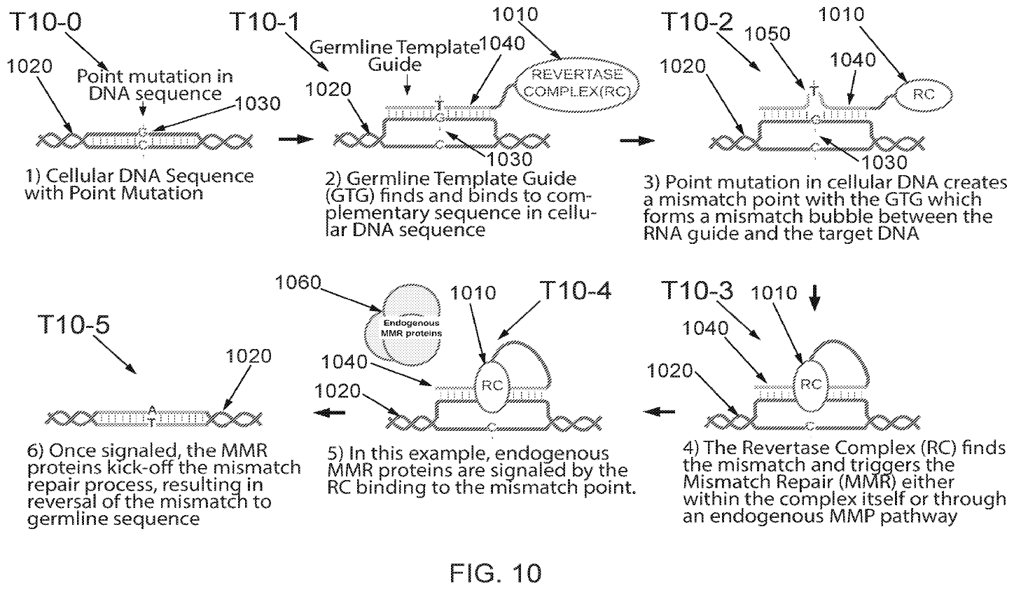

### FIG. 10

FIG. 10 illustrates a non-limiting example of a mechanism for the mismatch repair facilitation agent (e.g., RPC): (1) (See times T10-0 and T10-1.) A goal of the RPC (e.g., revertase complex 1010) is to trigger mismatch repair in the organism DNA 1020 while targeting it to the locus of interest (e.g., the point mutation 1030). (2) (See time T10-2.) To achieve this a guide 1040 is used to form a mismatch bubble 1050 at the site of mutation 1030. (3) (See time T10-3.) This mismatch bubble 1050 is then recognized by the RPC 1010 and flagged for repair. (4) (See times T10-3, T10-4 and T10-5.) Once flagged and signaled, the full MMR repair process can be carried out by either the endogenous MMR process that exists in the target organism (e.g., by endogenous MMR proteins 1060) or by the MMR complex 1010 itself.

In certain embodiments, instead of creating a cut in one of the DNA strands with Cas9 RuvC and/or other ss-nucleases, the other strand is left intact and bound to an extended guide RNA (gRNA), surfacing an existing mismatch.

### Guided MutSα

In certain embodiments, having a mismatch recognition protein in the near vicinity improves the speed and efficiency of the mutation repair. Therefore, MutSα is guided to the area that is being checked for mismatching by the PNA template. This aspect of the invention will be described in greater detail below.

To make a guided form of MutSα, a repeating peptide array termed SunTag, which can recruit multiple copies of an antibody-fusion protein, is fused to an inactive form of Cas9 (dCas9) (lacking a nuclear localization signal). A single-chain variable fragment (scFv) is fused to the MutSα subunit MSH6. In the cytosol the scFv binds to the SunTag, attaching the dCas9 to the MSH6; MSH6 dimerizes with MSH2 in the cytosol forming MutSα and is then imported to the nucleus. The nuclear localization of MSH6 is dependent on MSH2, and therefore dCas9 and the complete MutSα can be imported together.

In order to target a specific region, a guide is used. At least two types of guides are preferred: (1) The first one is a classical guide RNA that brings the dCas9-MutSα in close proximity to the mismatch control location, where the mismatch template is screening for mutations. (2) The second one is a PNA-RNA chimera guide. The guide goes to the location while being bound to the Cas9-MutSα, where the PNA is invading, and part of the RNA is checking for mismatches. Therefore, in this case, the PNA-RNA chimera is both the mismatch template and the guide. Upon mismatch formation, the MutSα can immediately recognize it and start the MMR process.

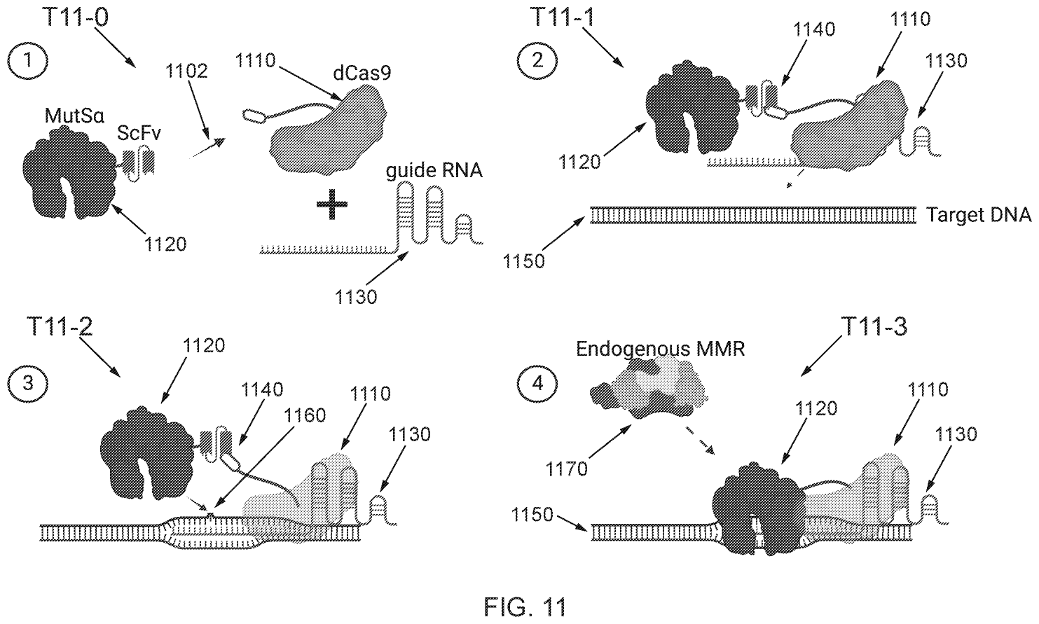

### FIG. 11

FIG. 11 illustrates dCas9 1110 and MutSα 1120, and an example mechanism of dCas9-MutSα guided by gRNA 1130 (1) At time T11-0, dCas9-MutSα forms a ribonucleoprotein 1140 with the mismatch guide 1130 (formation process denoted by arrow 1102). (2) At time T11-1, the mismatch guide 1130 directs the complex 1140 to the sequence-specific location 1150. (3) At time T11-2, the complex 1140 invades the organism DNA 1150 and the mismatch template 1130 checks for errors. (4) At time T11-3, mismatch bubbles 1160 are recognized by MutSα 1120 and the MMR repair cascade is initiated by MMR proteins 1170 (see arrow 1104).

### Comparison Agent

As discussed above, in preferred embodiments, the comparison agent is configured to bind to the first single-stranded organism DNA strand, and is so configured at least in part by including material that presents a number and configuration of hydrogen bonds of at least a portion of the second desired sequence (of the desired DNA sequence, see above).

That is, the change agent material can be any material that presents the number and configuration of hydrogen bonds of at least a portion of the second desired sequence (of the desired DNA sequence, see above). Stated alternatively, the change agent material can be material that chemically complements the first single-stranded organism DNA strand according to base pairing rules, such that the first single-stranded organism DNA strand will bind to the material in a manner sufficient to induce the indication of a mismatch if indeed a mismatch is present.

It should be understood that the number and configuration of hydrogen bonds presented need not be the number and configuration of hydrogen bonds of the entire second desired sequence (of the desired DNA sequence, see above). That is, the number and configuration of hydrogen bonds presented can be less than that of the entire second desired sequence (of the desired DNA sequence, see above). This enables, among other things, for the editing process of the present invention to provide for fixing multiple mismatches simultaneously or at least with the same dose. Further, this allows for the possibility that the number and configuration of hydrogen bonds presented for some unknown reason does not match that of the entire second desired sequence (of the desired DNA sequence, see above) exactly.

Accordingly, the comparison agent material can include one or more of the following presenting the second desired sequence number and configuration of hydrogen bonds: a sequence of nucleotide bases, a sequence of proteins, and a molecule.

Further accordingly, the comparison agent material can include one or more of the following complementary to the first single-stranded organism DNA strand: a single-stranded DNA strand, a double-stranded DNA strand, a single-stranded PNA strand, a double-stranded PNA strand, a single-stranded RNA strand, a double-stranded RNA strand, a strand including any permutation of any two or more of the foregoing, in series and/or parallel.

Therefore, in any of the illustrated examples, or other embodiments described herein, the PNA, RNA, and DNA instances of comparison agent material can be replaced by any other material that presents the second desired sequence number and configuration of hydrogen bonds, including but not limited to one or more of a PNA, a RNA, a DNA, a sequence of nucleotide bases, a sequence of proteins, and a molecule.

### Strand Invasion Agent

As discussed above, in preferred embodiments, the strand invasion agent is configured to cause a separation of a double-stranded DNA strand in at least one cell of the organism, the double-stranded DNA strand including the organism DNA sequence and comprising a first single-stranded organism DNA strand including the first current sequence (of the organism DNA sequence, see above) and a second single-stranded organism DNA strand bound to the first single-stranded organism DNA strand and including the second current sequence (of the organism DNA sequence, see above), the separation causing an unbinding of the first and second single-stranded organism DNA strands.

While PNA is used in the illustrated examples and discussed in several embodiments, any material that causes the separation can be used as the strand invasion agent, and the strand invasion agent is not limited to a PNA. Other materials for strand invasion can be DNA, RNA, guide RNA (gRNA), proteins, and other molecules. Full DNA denaturation can also occur through heat, salt, and NaOH (https://info.gbiosciences.com/blog/the-top-methods-for-dna-denaturation).

### Strand Invasion Agent Binding

In certain embodiments, the strand invasion agent is configured to bind to the second single-stranded organism DNA strand.

For example, the strand invasion agent can bind to the second single-stranded organism DNA strand while the comparison agent material binds to the first single-stranded organism DNA strand. This is shown in some of the illustrated examples.

Accordingly, in certain embodiments, the strand invasion agent includes binding material that presents a number and configuration of hydrogen bonds of at least a portion of the first current sequence.

That is, the strand invasion agent binding material can be any material that presents the number and configuration of hydrogen bonds of at least a portion of the first current sequence (of the organism DNA sequence, see above). Stated alternatively, the strand invasion agent binding material can be material that chemically complements the second single-stranded organism DNA strand according to base pairing rules. In some embodiments, the second single-stranded organism DNA strand should bind to the material in a manner sufficient to induce the indication of a mismatch if indeed a mismatch is present.

It should be understood that the number and configuration of hydrogen bonds presented need not be the number and configuration of hydrogen bonds of the entire first current sequence (of the organism DNA sequence, see above). That is, the number and configuration of hydrogen bonds presented can be less than that of the entire first current sequence (of the organism DNA sequence, see above). This enables, among other things, for the editing process of the present invention to provide for fixing multiple mismatches simultaneously or at least with the same dose. Further, this allows for the possibility that the number and configuration of hydrogen bonds presented for some unknown reason does not match that of the entire first current sequence (of the organism DNA sequence, see above) exactly.

Accordingly, the binding material can include one or more of the following presenting the first current sequence number and configuration of hydrogen bonds: a sequence of nucleotide bases, a sequence of proteins, and a molecule.

Further accordingly, the binding material can include one or more of the following complementary to the second single-stranded organism DNA strand: a single-stranded DNA strand, a double-stranded DNA strand, a single-stranded PNA strand, a double-stranded PNA strand, a single-stranded RNA strand, a double-stranded RNA strand, a strand including any permutation of any two or more of the foregoing, in series and/or parallel.

Therefore, in any of the illustrated examples, or other embodiments described herein, the PNA, RNA, and DNA instances of strand invasion agent material can be replaced by any other material that presents the first current sequence number and configuration of hydrogen bonds, including but not limited to one or more of a PNA, a RNA, a DNA, a sequence of nucleotide bases, a sequence of proteins, and a molecule.

### Strand Bias and Methylation

As discussed above, in certain embodiments, one or more of the strand invasion agent and the comparison agent material include features facilitating direction of the mismatch repair process to the organism DNA strand(s) and/or causing a bias of the mismatch repair process toward the organism DNA strand(s), the features including one or more of pseudo-complementary base pairs, and methylation modifications.

These features assist in directing the mismatch repair to the correct segment (i.e., to the organism DNA strand and not the comparison agent (e.g., guide)). In certain embodiments, these features alter the comparison agent (e.g., guide) to bias the MMR reaction away from altering the comparison agent versus altering the targeted organism DNA strand(s). Certain features include methylation and/or other modification necessary to bias the strand repair to the correct segment.

In certain embodiments, pseudo-complementary base pairs (that prefer to bind to DNA rather than PNA) are used for one or both of the strand invasion agent and the change agent.

At least one purpose for such use is to avoid the invasion (part of the) strand and the comparison (part of the) strand binding to each other instead of binding to the target strand. The pseudo-complementary base pairs are formally sequence-complementary, but have significantly reduced affinity for forming duplexes with each other due to chemical modification. Pseudo-complementary base pairing can be for example achieved by substituting the following base pairs: adenine by 2,6-diaminopurine, thymine (or uracil) by 2-thiouracil (or 2-thiothymine)(1), guanine N6-methoxy-2,6-diaminopurine, and cytosine by and N4-benzoylcytosine (2). There are however other modifications possible and existing to create such pseudo-complementary base pairs e.g., 7-nitro-7-deazahypoxanthine and 2-thiocytosine (3).

In certain embodiments, in order to encourage DNA repair machinery to discriminate between comparison agent and target DNA strand and bias toward the target DNA when deciding which side of the mismatch to repair, the comparison strand will be modified to bias away from itself. These modifications can include methylation of the nucleobases. Methylation of nucleobases could be (but is not limited to) N6-Methyladenine, 5-Methylcytosine, N4-Methylcytosine, 5-methyluracil, n7-methylguanosine.

Further types of modification to the comparison strand can be modification to the comparison strand backbone e.g. using phosphorothioates, methylphosphonates, or modifications on the 2′-sugar position.

### Increasing Separation Efficiency

In certain embodiments, one or both of the strand invasion agent and the change agent material has at least one property that increases efficiency of the separation.

In certain embodiments, the at least one property is one or more of (1) Increased binding strength between strand invasion agent and targeted strand (and/or between change agent and targeted strand), so that it “sticks” to the target sequence longer, (2) increased energetic favorability between change agent and targeted strand versus targeted strand and its complementary existing strand, (3) lower energy required for unbinding, (4) higher energy requirement to bind, in order to increase accuracy (since it would need higher sequence complementarity to have enough energy to bind), (5) modification to bias it against the comparison material and towards the targeted strand, (6) changes to increase stability during delivery, (7) changes to increase ease of removal by cell, (8) changes to decrease removal by cell, and (9) changes to increase/decrease solubility.

For example without limitation, it is possible to increase or decrease the binding strength of the comparison agent material as to a given target DNA strand, such that it “sticks” on the target DNA strand more or less strongly. This can be important, for example without limitation, for safety because if it is too tightly bound the body might assume it is toxic and cut out the DNA entirely, causing damage. The binding efficiency is most preferably in the “goldilocks” zone: tight enough to give the comparison agent time to compare, and loose enough to unbind and let the cell continue functioning. This is also important, for example without limitation, for multiplex editing (e.g., conducting multiple edits to the DNA strand at one time).

In certain embodiments, one or both of the strand invasion agent and the change agent material comprise a compound having a chemical structure that provides the at least one property.

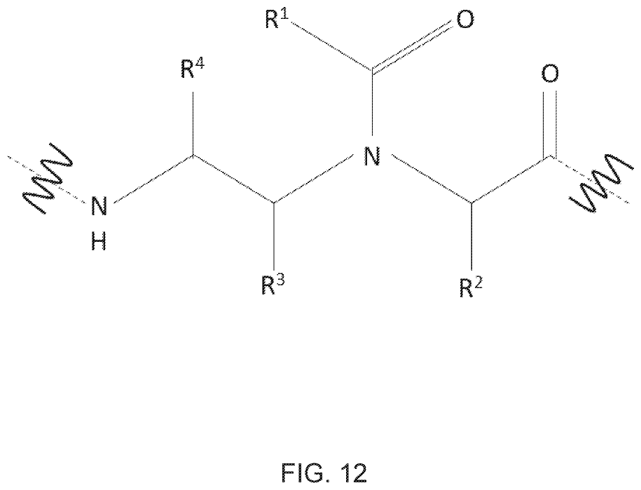

A preferred chemical structure of a strand invasion agent is illustrated in FIG. 12. The illustrated structure forms an oligomeric sequence comprising a repeating unit having the formula illustrated in FIG. 12, wherein R1 is an alkyl group covalently bound to a nucleobase, R2-4 group is a hydrogen group, and the at least one property results from modifying one or more of the R groups to have one or more of the following, combinatorially: amide group, ketone, alkyl, O-alkyl.

The illustrated structure forms a PNA that is useful and effective for the purposes described herein with reference to PNAs. The illustrated PNA is useful and effective even with no modifications (e.g., the R groups are not modified), but modification of the R groups as indicated can cause the PNA to have properties (those set forth above, and others) that increase the efficiency of the separation.

Such variations can include but are not limited to the following:

(1) Where R¹ is alkyl or O-alkyl, any of which is unsubstituted or substituted; and n is i, 2, 3, 4, 5, 6, 7, 8, 9, or 10, or a pharmaceutically-acceptable salt or ionized form thereof, and R², R³, and R⁴ is a hydrogen(H).

(2) Where R³ and R⁴ is a hydrogen, and R² an amide group, ketone, alkyl, O-alkyl any of which is unsubstituted or substituted; and n is i, 2, 3, 4, 5, 6, 7, 8, 9, or 10, or a pharmaceutically-acceptable salt or ionized form thereof.

(3) Where R² and R⁴ are hydrogens, and R³ an amide group, ketone, alkyl, O-alkyl any of which is unsubstituted or substituted; and n is i, 2, 3, 4, 5, 6, 7, 8, 9, or 10, or a pharmaceutically-acceptable salt or ionized form thereof.

(4) Where R² and R³ are hydrogens and R⁴ an amide group, ketone, alkyl, O-alkyl any of which is unsubstituted or substituted; and n is i, 2, 3, 4, 5, 6, 7, 8, 9, or 10, or a pharmaceutically-acceptable salt or ionized form thereof.

(5) The compound in any one of (1), (3) and (4) where R² is a linear alkyl.

(6) The compound in any one of (1), (3) and (4) where R² is a methyl.

(7) The compound in any one of (1), (2) and (4) where R³ is a linear alkyl.

(8) The compound in any one of (1), (2) and (4) where R³ is a methyl.

(9) The compound in any one of (1), (2) and (3) where R⁴ is a linear alkyl.

(10) The compound in any one of (1), (2) and (3) where R⁴ is a methyl.

(11) The compound in any one of (3), (7) and (8) where R² is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(12) The compound in any one of (4), (9) and (10), where R² is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(13) The compound in any one of (2), (5) and (6), where R³ is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(14) The compound in any one of (4), (9) and (10), where R³ is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(15) The compound in any one of (2), (5) and (6), where R⁴ is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(16) The compound in any one of (3), (7) and (9), where R⁴ is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

(17) The compound in (11) where R⁴ is an amide group, ketone, alkyl, methyl, O-alkyl any of which is unsubstituted or substituted; and n is i, 2, 3, 4, 5, 6, 7, 8, 9, or 10, or a pharmaceutically-acceptable salt or ionized form thereof.

The oligonucleotide preferably has a length between 10-100, but can be longer.

Preferably, on R¹ is the area where the nucleobases will be attached. The present invention contemplates the attachment of any nucleobase and any modifications thereto, including but not limited to adenine, guanine, thymine, uracil, cytosine, methylated-cytosine (5mC, 4mC), methylated-adenine (6 mA) 2-thiouracil, 2-aminopurine, methoxy-2,6-diaminopurine, N4-benozylcytosine, N7-methylguanin, and any other purine and pyrimidine derivative.

### Testing Data

As noted above, the illustrated structure forms a PNA that is useful and effective for the purposes described herein with reference to PNAs. The illustrated PNA is useful and effective even with no modifications (e.g., the R groups are not modified).

The illustrated PNA has been tested in accordance with the method of the present invention, and data has been obtained showing it successfully invading and displacing DNA that is in a duplex double-stranded form.

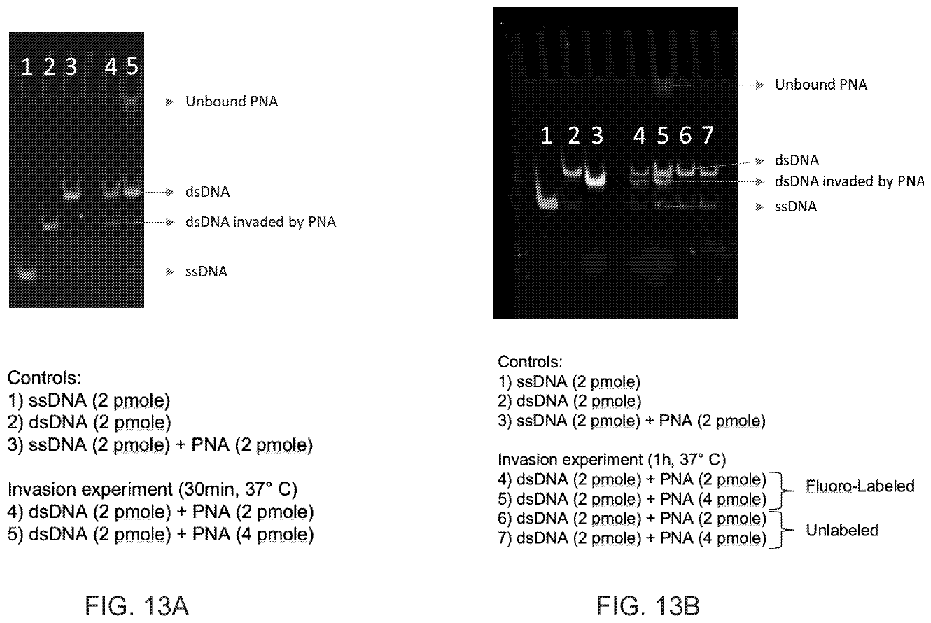

With regard to the conducted testing, tested was invasion of double-stranded DNA by a PNA strand. More specifically, referring now to FIGS. 13A and 13B, in two experiments A (FIG. 13A) and B (FIG. 13B), electrophoretic mobility shift assay was performed to determine whether a 20mer PNA can invade and kick off the double-strand DNA (dsDNA), either with 20mer dsDNA (Experiment A) or 40mer dsDNA (Experiment B). It was determined that the complementary PNA can bind to the single-strand DNA (ssDNA)(A&B: lane 3) and it efficiently invades dsDNA (A&B: lane 4 and 5). In addition, the PNA not only invades but can kick off the 2mer DNA (A: lane 4 and 5). PNA that is not bound to DNA does not run into the gel, because of its charge (A&B: lane 5). All DNA is labeled with a red dye, the fluorescent labeled PNA is visualized in green. Control details and experiment parameters are indicated on FIGS. 13A and 13B.

### Acronyms and Terms

The following acronyms and terms as used herein are understood to have the following definitions, as complementary and/or supplementary to (and not as a replacement for) their definitions as known in the relevant art and/or to a skilled artisan in the relevant art:

CRISPR (Clustered Regular Interspaced Short Palindromic Repeats): a genetic engineering tool that uses a CRISPR sequence of DNA and its associated protein to edit the base pairs of a gene.

DNA (Deoxyribonucleic Acid): a self-replicating material present in nearly all living organisms as the main constituent of chromosomes. It is the carrier of genetic information.

RNA (Ribonucleic Acid): a nucleic acid present in all living cells. Its principal role is to act as a messenger carrying instructions from DNA for controlling the synthesis of proteins, although in some viruses RNA rather than DNA carries the genetic information.

ssODN (single-stranded Oligodeoxynucleotide): a short sequence of nucleotides, whose nucleotides contain deoxyribose.

RNP (ribonucleoprotein): a nucleoprotein that contains RNA.

DSB (double-strand break): a break in the DNA in which both strands in the double helix are severed.

sgRNA (single guide RNA): a chimera of crRNA and tracrRNA that is typically 100 nucleotides in length and consists of three regions: (a) a user defined, 17-20nt base-pairing region for specific DNA binding; (b) a 40nt Cas9 handle hairpin for Cas9 protein binding; and (c) a 40nt long transcription terminator derived from S. pyogenes, that contains hairpin structures that provide stability to the RNA molecule.

Cas (CRISPR associated genes): RNA-guided DNA endonuclease enzyme associated with the CRISPR adaptive immunity system in Streptococcus pyogenes, among other bacteria.

NHEJ (Non-homologous end joining): a pathway that repairs double-strand breaks in DNA. NHEJ is referred to as “non-homologous” because the break ends are directly ligated without the need for a homologous template, in contrast to homology directed repair, which requires a homologous sequence to guide repair.

BER (Base Excision Repair): a cellular mechanism that repairs damaged DNA throughout the cell cycle. It is responsible primarily for removing small, non-helix-distorting base lesions from the genome.

DN (Double Nick): a discontinuity in a double stranded DNA molecule where there is no phosphodiester bond between adjacent nucleotides of both strands of the DNA molecule, typically through damage or enzyme action.

SN (Single Nick): A nick is a discontinuity in a double stranded DNA molecule where there is no phosphodiester bond between adjacent nucleotides of one strand typically through damage or enzyme action.

crRNA (CRISPR RNA): contains variable targeting sequence required for the Cas9 protein to target the DNA strand. crRNA forms a complex with tracrRNA to allow the Cas9 protein to bind to and cleave the DNA strand.

SaCas9 (Staphylococcus aures Cas9): Cas9 homologue found natively in Staphylococcus aureus bacteria.

SpCas9 (Staphylococcus pyogenes Cas9): Cas9 homologue found natively in Staphylococcus pyogenes bacteria.

tracrRNA (Trans-activating crRNA): a small trans-encoded RNA. It was first discovered in the human pathogen Streptococcus pyogenes. TracrRNA is complementary to and base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

nDNA (Nuclear deoxyribonucleic acid): the DNA contained within the nucleus of a eukaryotic organism. Nuclear DNA encodes for the majority of the genome in eukaryotes, with mitochondrial DNA and plastid DNA coding for the rest.

AAV (Adeno-associated Virus): a small virus which infects humans and some other primate species. AAV is not currently known to cause disease. The virus causes a very mild immune response, lending further support to its apparent lack of pathogenicity. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise.

Donor sequence: Also referred to sometimes as a repair sequence, this is a sequence of DNA in, e.g., a homologous repair template, that is to replace a DNA sequence to be changed, e.g., as a result of a CRISPR event.

Intercalator: “DNA intercalators include aromatic heterocyclic compounds of various chemical classes with profound biological activities. The flat molecules of these ligands intercalate between base pairs of DNA right-handed helix, lengthening and unwinding this structure at the intercalation sites. Besides, other physico-chemical criteria of DNA intercalation are as following: the increase in the contour length of duplex DNA; unwinding of supercoils from natural supercoiled covalently closed duplex DNA; the increase in Tm of DNA in the complexes with ligands.” (from https://pubmed.ncbi.nlm.nih.gov/1814033/).

### Preparing the Treatment

In preferred embodiments, preparing the treatment comprises preparing the change agent material.

Further in preferred embodiments, preparing the change agent material comprises preparing the strand invasion agent. In certain embodiments, in which the strand invasion agent incorporates a PNA, preparation of the strand invasion agent comprises synthesis of a PNA.

Preferably, the strand invasion agent is prepared using any effective technique, now known or hereafter developed. Such techniques can include, but are not limited to, for strand invasion agents incorporating PNA, the PNA synthesis methods described at the following link: https://pna.creative-peptides.com/services/pna-synthesis.html.

Further in preferred embodiments, preparing the change agent material comprises preparing the comparison agent. In certain embodiments, in which the comparison agent incorporates a DNA, preparation of the strand invasion agent comprises synthesis of a DNA.

Preferably, the comparison agent is prepared using any effective technique, now known or hereafter developed. Such techniques can include, but are not limited to, for comparison agents incorporating DNA, the DNA synthesis methods described at the following link: https://synbio-tech.com/dna-synthesis-definition-and-methods/.

Further with regard to the preparation of the comparison agent, the preparation of the comparison agent can comprise one or more of the following steps:

(1) sequence target cellular DNA to get the full germline sequence; (2) determine or otherwise decide on a desired length (e.g., size; base pair number; etc.) that will be the length of each comparison agent material sequence (e.g., guide sequence) that will be used for comparison against (e.g., mismatch detection on) a corresponding portion of the organism's current DNA; this length will be referenced here as “L”; (3) using L, determine or otherwise decide on a desired length (e.g., size; base pair number; etc.) of overlap (this length will be referenced here as “O”) to establish, when using multiple adjacent sequences to cover a portion of the genome longer than L, between the adjacent sequences (e.g., the first guide sequence spans from position 1 to position 35, and a second guide sequence spans from position 30 to position 65; these example guide sequences have an O of 5, i.e., 5 nucleotide bases of overlap). (4) generate the comparison agent material guide sequences, as follows: For guide sequence 1, determine or otherwise decide on a starting location/position X₁ on the organism DNA sequence/strand; X₁+L is then the ending location/position of the guide sequence 1. X₂=(X₁+L)-O is then the starting location/position of the next guide sequence. Continue this process (with X_{(S+1)}=(X_{S}+L)-O being a starting position and X_{(S+1)}+L being an ending position of each of the comparison agent material sequences (S=0 to n)) until a desired area of the organism DNA sequence/strand is covered. (If O=0 the sequences are back to back with no overlap.)

### Applying the Treatment

In preferred embodiments, applying the treatment comprises delivering the change agent material to one or more cells of the organism by one or more of the following processes: injection, intra venous, intra muscular, intra ocular, intra peritoneal, intra-cranial, topical, aerosolized spray, oral liquid, oral pill, transdermal patch, ex-vivo alteration and re-injection.

It should be understood that any suitable methods, now known or hereafter developed, can be used to apply the treatment.

### BIBLIOGRAPHY

The following references and any other references and links discussed or set forth herein are incorporated by reference in their entireties.
1. Mismatch repair pathway: molecules, functions, and role in colorectal carcinogenesis. Aga Syed Sameera, Saniya Nissarb and Kaneez Fatimac. European Journal of Cancer Prevention 23:246-257 c 2014 Wolters Kluwer Health | Lippincott Williams & Wilkins.
2. Kunkel T A, Erie D A (2005). DNA mismatch repair. Annu Rev Biochem 74:681-710.
3. Li G-M (2008). Mechanisms and functions of DNA mismatch repair. Cell Res 18:85-98.
4. Kamiya, Y., Sato, F., Murayama, K., Kodama, A., Uchiyama, S., & Asanuma, H. (2020). Front Cover: Incorporation of Pseudo-complementary Bases 2,6-Diaminopurine and 2-Thiouracil into Serinol Nucleic Acid (SNA) to Promote SNA/RNA Hybridization (Chem. Asian J. 8/2020). Chemistry-An Asian Journal, 15(8), 1180-1180.
5. Olsen, A. G., Dahl, O., Petersen, A. B., Nielsen, J., & Nielsen, P. I. (2011). A novel pseudo-complementary PNA GC base pair. Artificial DNA: PNA & XNA, 2(1), 32-36.
6. Lahoud, C., Timoshchuk, V., Lebedev, A., Arar, K., Hou, Y. M., & Gamper, H. (2008). Properties of pseudo-complementary DNA substituted with weakly pairing analogs of guanine or cytosine. Nucleic acids research, 36(22), 6999-7008.
7. Faddeeva M D, Beliaeva T N. DNK-interkaliatory: vzaimodeistvie s DNK i drugimi kletochnymi komponentami i primenenie v biologicheskikh issledovaniiakh [DNA intercalators: their interaction with DNA and other cell components and their use in biological research]. Tsitologiia. 1991; 33(10):3-31. Russian. PMID: 1814033.
8. Koppitz, M., Nielsen, P. E., & Orgel, L. E. (1998). Formation of Oligonucleotide-PNA-Chimeras by Template-Directed Ligation. Journal of the American Chemical Society, 120(19), 4563-4569. doi:10.1021/ja974190y.
9. Ray, A. and Nordén, B. (2000), Peptide nucleic acid (PNA): its medical and biotechnical applications and promise for the future. The FASEB Journal, 14: 1041-1060. https://doi.org/10.1096/fasebj.14.9.1041.

### Additional Aspects of the Invention

It should be understood that the present invention encompasses the use of the described systems, devices and methods for partial or whole genome replacement in one or more cells of an organism, and not only in the medical fields, but also in other fields, and that the present invention is not limited to DNA sequence replacement, and that applications of the present invention to DNA sequence replacement are merely a subset of the possible embodiments of the present invention.

It should be understood that one or more systems and methods of the present invention preferably can be integrated with existing systems and methods and that any of such systems and methods of the present invention can be applied to effect partial or whole genome replacement in connection with one or more aspects of such systems and methods.

It should be understood that while certain methods are discussed herein as including preferred steps in a preferred order, it is contemplated that the present invention encompasses the steps being accomplished in other orders, not all of the steps being necessary, and/or one or more additional steps being taken, without departing from the scope of the present invention.

It should be understood that systems and methods described herein can be, but are not required to be, accomplished with or without the use of machines (including, but not limited to, with or without computers), and/or by one or more engines (such engines preferably including software running on at least one computer machine with a processor, memory, data storage capability, and networking capability, and preferably on two or more such computer machines communicating over a network such as, for example, the Internet), and, in certain embodiments, accomplished remotely, that is, over a network such as, for example, the Internet, an intranet, a wide area network, a local area network, and/or other network, through the operation of machines communicating with one another over the network, such as, for example, computers, tablets, smartphones, appliances, or any other network-enabled device. Further, calculations, transmissions and storage needed and/or employed by the present invention can be, but are not required to be, accomplished with secure calculation, transmission and storage protocols and using encryption and decryption protocols.

It should be understood that the headings set forth herein are non-limiting and provided for convenience. It should be understood that while arrows and lines on the drawings have in certain instances been described herein as representing one direction of communication and/or activity, the present invention encompasses embodiments in which such lines of communication and/or activity are bi-directional or in the opposite direction than as indicated. Unless otherwise defined, all technical and scientific terms used herein have the same meaning as commonly understood by one of ordinary skill in the art. Preferred methods and materials are described herein, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not. It should be understood that descriptions of embodiments, examples and instances of the present invention set forth, and any and all aspects thereof are non-limiting and that the present invention encompasses at least the broadest concepts brought to light by the present disclosure.

What is claimed is:

1. A method of changing a DNA sequence of an organism, the organism DNA sequence comprising a first current sequence of nucleotide bases and a second current sequence of nucleotide bases matched by base pairing rules to the first current sequence of nucleotide bases to form a respective base pair of complementary nucleotide bases at each of one or more locations in the organism DNA sequence, the method comprising the steps of:
determining a desired DNA sequence, the desired DNA sequence being a DNA sequence to which it is desired that the DNA sequence of the organism be changed, the desired DNA sequence comprising a first desired sequence of nucleotide bases and a second desired sequence of nucleotide bases matched by the base pairing rules to the first desired sequence of nucleotide bases to form a respective base pair of complementary nucleotide bases at each of one or more locations in the desired DNA sequence;
preparing a treatment configured to cause the organism DNA sequence to be changed to the desired DNA sequence; and
applying the treatment to the organism; wherein
the treatment is configured to cause the organism DNA sequence to be changed to the desired DNA sequence by causing a changing of one or both of the nucleotide bases at each of one or more of the organism DNA sequence locations to the nucleotide bases at a corresponding desired DNA sequence location;
applying the treatment includes delivering to the organism at least one dose; and
each dose includes respective change agent material that causes the changing; and wherein
the change agent material includes:
a strand invasion agent configured to cause a separation of a double-stranded DNA strand in at least one cell of the organism, the double-stranded DNA strand including the organism DNA sequence and comprising a first single-stranded organism DNA strand including the first current sequence and a second single-stranded organism DNA strand bound to the first single-stranded organism DNA strand and including the second current sequence, the separation causing an unbinding of the first and second single-stranded organism DNA strands; and
a comparison agent configured to bind to the first single-stranded organism DNA strand, the comparison agent being so configured at least in part by including material that presents a number and configuration of hydrogen bonds of at least a portion of the second desired sequence; and wherein
when the comparison agent material binds to the first single-stranded organism DNA strand and at least one mismatched base pair is indicated on the first single-stranded organism DNA strand as a result of the binding, the organism initiates a mismatch repair process.

2. The method of claim 1, wherein the change agent material further includes a mismatch repair facilitation agent configured to facilitate the mismatch repair process, the mismatch repair facilitation agent including one or more of the following molecules: hMut∝, hMutSβ, hMutL∝, hMutLβ, hMutLγ, Exol, Pol δ, PCNA, RPA, HMGB1, RFC, DNA Ligase I, Cas9 RuvC domain (alone or in combination with a guide RNA), Cas9 HNH domain, single-strand DNA (ssDNA) nuclease, deactivated Cas9 (dCas9) fusion with one or more mismatch repair proteins, CRISPR (or other editor) variants that allow for search, selection and/or repair of somatic mutations.

3. The method of claim 1, wherein one or more of the strand invasion agent and the comparison agent material include features causing a bias of the mismatch repair process toward the first single-stranded organism DNA strand, the features including one or more of pseudo-complementary base pairs, and methylation modifications.

4. The method of claim 1, wherein one or both of the strand invasion agent and the change agent material has at least one property that increases efficiency of the separation, the property being one or more of: increased binding strength, increased energetic favorability, lower energy required for unbinding, higher energy requirement to bind, modification biasing against, increase stability during delivery, increase ease of removal by organism cell, decrease removal by organism cell, increase solubility, decrease solubility.

5. The method of claim 4, wherein one or both of the strand invasion agent and the change agent material comprise a compound having a chemical structure that provides the at least one property.

6. The method of claim 5, wherein the chemical structure forms an oligomeric sequence comprising a repeating unit having the following formula: 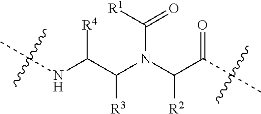
wherein each R1 is an alkyl group covalently bound to a nucleobase, R2-4 group is a hydrogen group, and the at least one property results from modifying one or more of the R groups to have one or more of the following, combinatorially: amide group, ketone, alkyl, O-alkyl.

7. The method of claim 1, wherein the strand invasion agent is configured to bind to the second single-stranded organism DNA strand.

8. The method of claim 7, wherein the strand invasion agent includes binding material that presents a number and configuration of hydrogen bonds of at least a portion of the first current sequence.

9. The method of claim 8, wherein the binding material includes one or more of the following presenting the first current sequence number and configuration of hydrogen bonds: a sequence of nucleotide bases, a sequence of proteins, and a molecule.

10. The method of claim 9, wherein the binding material includes one or more of the following complementary to the second single-stranded organism DNA strand: a single-stranded DNA strand, a double-stranded DNA strand, a single-stranded PNA strand, a double-stranded PNA strand, a single-stranded RNA strand, a double-stranded RNA strand, a strand including any permutation of any two or more of the foregoing, in series and/or parallel.

11. The method of claim 1, wherein the comparison agent material includes one or more of the following presenting the second desired sequence number and configuration of hydrogen bonds: a sequence of nucleotide bases, a sequence of proteins, and a molecule.

12. The method of claim 11, wherein the comparison agent material includes one or more of the following complementary to the first single-stranded organism DNA strand: a single-stranded DNA strand, a double-stranded DNA strand, a single-stranded PNA strand, a double-stranded PNA strand, a single-stranded RNA strand, a double-stranded RNA strand, a strand including any permutation of any two or more of the foregoing, in series and/or parallel.

13. The method of claim 1, wherein the desired DNA sequence is at least a portion of one or more of a germline DNA sequence of the organism and a DNA sequence differing from the organism DNA sequence with respect to only one base pair.

14. The method of claim 13, wherein the desired DNA sequence is the germline DNA sequence of the organism and determining the desired DNA sequence comprises sequencing one or more cells of the organism.

15. The method of claim 1, wherein preparing the treatment comprises preparing one or both of the strand invasion agent and the comparison agent, the preparation of the strand invasion agent comprising synthesis of a PNA, the preparation of the comparison agent comprising synthesis of DNA strands in accordance with the following steps: determine a sequence length L; determine an overlap length O; generate a plurality of adjacent comparison agent material sequences, using a formula referencing a starting position X on the second desired sequence, with X_{(S+1)}=(X_{S}+L)-O being a starting position and X_{(S+1)}+L being an ending position of each of the comparison agent material sequences (S=0 to n), until the second desired sequence is covered by the DNA strands.

16. The method of claim 1, wherein applying the treatment comprises delivering the change agent material to one or more cells of the organism by one or more of the following processes: injection, intra venous, intra muscular, intra ocular, intra peritoneal, intra-cranial, topical, aerosolized spray, oral liquid, oral pill, transdermal patch, ex-vivo alteration and re-injection.

17. A compound for changing a DNA sequence of an organism to a desired DNA sequence, the organism DNA sequence comprising a first current sequence of nucleotide bases and a second current sequence of nucleotide bases matched by base pairing rules to the first current sequence of nucleotide bases to form a respective base pair of complementary nucleotide bases at each of one or more locations in the organism DNA sequence, the desired DNA sequence being a DNA sequence to which it is desired that the DNA sequence of the organism be changed, the desired DNA sequence comprising a first desired sequence of nucleotide bases and a second desired sequence of nucleotide bases matched by the base pairing rules to the first desired sequence of nucleotide bases to form a respective base pair of complementary nucleotide bases at each of one or more locations in the desired DNA sequence, the compound comprising:
change agent material configured to cause the organism DNA sequence to be changed to the desired DNA sequence by causing a changing of one or both of the nucleotide bases at each of one or more of the organism DNA sequence locations to the nucleotide bases at a corresponding desired DNA sequence location, the change agent material including:
a strand invasion agent configured to cause a separation of a double-stranded DNA strand in at least one cell of the organism, the double-stranded DNA strand including the organism DNA sequence and comprising a first single-stranded organism DNA strand including the first current sequence and a second single-stranded organism DNA strand bound to the first single-stranded organism DNA strand and including the second current sequence, the separation causing an unbinding of the first and second single-stranded organism DNA strands; and
a comparison agent configured to bind to the first single-stranded organism DNA strand, the comparison agent being so configured at least in part by including material that presents a number and configuration of hydrogen bonds of at least a portion of the second desired sequence; such that
when the comparison agent material binds to the first single-stranded organism DNA strand and at least one mismatched base pair is indicated on the first single-stranded organism DNA strand as a result of the binding, the organism initiates a mismatch repair process.

18. The compound of claim 17, wherein one or both of the strand invasion agent and the change agent material has at least one property that increases efficiency of the separation, the property being one or more of: increased binding strength, increased energetic favorability, lower energy required for unbinding, higher energy requirement to bind, modification biasing against, increase stability during delivery, increase ease of removal by organism cell, decrease removal by organism cell, increase solubility, decrease solubility.

19. The method of claim 18, wherein one or both of the strand invasion agent and the change agent material comprise a compound having a chemical structure that provides the at least one property.

20. The method of claim 19, wherein the chemical structure forms an oligomeric sequence comprising a repeating unit having the following formula: 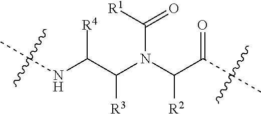
wherein R1 is an alkyl group covalently bound to a nucleobase, R2-4 group is a hydrogen group, and the at least one property results from modifying one or more of the R groups to have one or more of the following, combinatorially: amide group, ketone, alkyl, O-alkyl.
